SEQUENCE LISTING (53 sequences)

SEQ ID NO: 1 - protein, 509 aa, synthetic construct
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 2 - protein, 26 aa, synthetic construct
MATGSRTSLLLAFGLLCLPWLQEGSA

SEQ ID NO: 3 - protein, 18 aa, synthetic construct
MKWVTFISLLFLFSSAYS

SEQ ID NO: 4 - protein, 21 aa, synthetic construct
MAAHLLPICALFLTLLDMAQG

SEQ ID NO: 5 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLGPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 6 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTSGALLCSQALCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 7 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLGPFILNVTSGALLCSQALCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 8 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWVSWENTRTKESCQAIKEYMDTTLGPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 9 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 10 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWVSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 11 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSNTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 12 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 13 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 14 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 15 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTSGAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 16 - protein, 453 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 17 - protein, 430 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCI

SEQ ID NO: 18 - protein, 433 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 19 - protein, 436 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKP

SEQ ID NO: 20 - protein, 452 aa, synthetic construct
FRGPLLPNRPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 21 - protein, 452 aa, synthetic construct
FRGPLLPNRPFTTVWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 22 - protein, 433 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLK

SEQ ID NO: 23 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 24 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 25 - protein, 451 aa, synthetic construct
APPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 26 - protein, 449 aa, synthetic construct
PVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 27 - protein, 432 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 28 - protein, 429 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVC

SEQ ID NO: 29 - protein, 426 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIAD

SEQ ID NO: 30 - protein, 423 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVC

SEQ ID NO: 31 - protein, 420 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAV

SEQ ID NO: 32 - protein, 433 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLK

SEQ ID NO: 33 - protein, 435 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPP

SEQ ID NO: 34 - protein, 436 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPM

SEQ ID NO: 35 - protein, 437 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPME

SEQ ID NO: 36 - protein, 438 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMET

SEQ ID NO: 37 - protein, 439 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETE

SEQ ID NO: 38 - protein, 454 aa, synthetic construct
NFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 39 - protein, 452 aa, synthetic construct
RAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 40 - protein, 450 aa, synthetic construct
PPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 41 - protein, 428 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCI

SEQ ID NO: 42 - protein, 429 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCID

SEQ ID NO: 43 - protein, 430 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 44 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 45 - protein, 441 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 46 - protein, 443 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 47 - protein, 445 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 48 - protein, 447 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNA

SEQ ID NO: 49 - protein, 449 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASP

SEQ ID NO: 50 - protein, 451 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPST

SEQ ID NO: 51 - protein, 435 aa, synthetic construct
MAAHLLPICALFLTLLDMAQGFRGPLLPNRPFTTVWNANTQWCLERHGVDVDVSVFDVVANPGQTFRGPDMTIFYSSQLGTYPYYTPTGEPVFGGLPQNASLIAHLARTFQDILAAIPAPDFSGLAVIDWEAWRPRWAFNWDTKDIYRQRSRALVQAQHPDWPAPQVEAVAQDQFQGAARAWMAGTLQLGRALRPRGLWGFYGFPDCYNYDFLSPNYTGQCPSGIRAQNDQLGWLWGQSRALYPSIYMPAVLEGTGKSQMYVQHRVAEAFRVAVAAGDPNLPVLPYVQIFYDTTNHFLPLDELEHSLGESAAQGAAGVVLWVSWENTRTKESCQAIKEYMDTTLGPFILNVTSGALLCSQALCSGHGRCVRRTSHPKALLLLNPASFSIQLTPGGGPLSLRGALSLEDQAQMAVEFKCRCYPGWQAPWCERKSMW

SEQ ID NO: 52 - protein, 9 aa, synthetic construct
FRGPLLPNR

SEQ ID NO: 53 - protein, 14 aa, synthetic construct
FRGPLLPNRPFTTV

(19) United States
(12) Patent Application Publication
(10) Pub. No.: US 2026/0297215 A1
(43) Pub. Date: Oct. 1, 2026

(54) PHARMACEUTICAL COMPOSITION FOR SUBCUTANEOUS ADMINISTRATION CONTAINING HUMAN HYALURONIDASE PH20 VARIANT AND DRUG

(71) Applicant: ALTEOGEN Inc., Daejeon (KR)

(72) Inventors: Soon Jae PARK, Daejeon (KR); Hye-Shin CHUNG, Daejeon (KR); Seung Joo LEE, Daejeon (KR); Kyuwan KIM, Daejeon (KR); Minsoo BYUN, Daejeon (KR); Ki Seok NAM, Daejeon (KR); Sun-Ah YOU, Daejeon (KR); Chang Woo LEE, Daegu (KR); Hyung-Nam SONG, Daejeon (KR)

(73) Assignee: ALTEOGEN Inc., Daejeon (KR)

(21) Appl. No.: 19/532,992

(22) Filed: Feb. 6, 2026

Related U.S. Application Data
(63) Continuation of application No. 17/052,952, filed on Mar. 24, 2020, now Pat. No. 12,570,766.

(30) Foreign Application Priority Data
Mar. 25, 2019 (KR) 10-2019-0033880

(51) Int. Cl. C07K 16/32; A61K 45/06; A61K 47/26; C12N 9/26

(52) U.S. Cl. CPC C07K 16/32; A61K 45/06; A61K 47/26; C12N 9/2474; C07K 2317/94; C12Y 302/01035

(57) ABSTRACT

The present invention relates to a pharmaceutical composition including (a) a drug and (b) a human PH20 variant. The human PH20 variant included in the pharmaceutical composition according to the present invention comprises amino acid residue substitution(s) in one or more regions selected from an alpha-helix 8 region (S347 to C381) and a linker region (A333 to R346) between alpha-helix 7 and alpha-helix 8 in wild-type human PH20 having the amino acid sequence of SEQ ID NO: 1, wherein amino acid residue(s) located at the N-terminus or the C-terminus is(are) selectively truncated. In addition, the pharmaceutical composition according to the present invention may further comprise a pharmaceutically acceptable additive, particularly a stabilizer. The pharmaceutical composition according to the present invention can maximize the therapeutic effect of a drug used in combination therewith, due to the effect of human PH20 variants.

### CROSS-REFERENCE TO RELATED APPLICATIONS

This application is a continuation of U.S. patent application Ser. No. 17/052,952, filed Nov. 4, 2020, now U.S. Patent No. 12, 570, 766, which is a U.S. National Stage Application under 35 U.S.C. § 371 of International Patent Application No. PCT/KR2020/003975, filed Mar. 24, 2020, which in turn claims the benefit of priority under 35 U.S.C. § 119 to Korean Patent Application No. 10-2019-0033880, filed Mar. 25, 2019, the disclosure of each of which is incorporated by reference herein in its entirety.

### REFERENCE TO SEQUENCE LISTING SUBMITTED VIA PATENT CENTER

This application contains an electronic Sequence Listing which has been submitted in XML file format via Patent Center, the content of which is incorporated by reference herein in its entirety. The Sequence Listing XML file submitted via Patent Center is entitled “14463-742-999 SEQ LISTING. xml”, was created on Jan. 17, 2026, and is 67,815 bytes in size.

### TECHNICAL FIELD

The present invention relates to a pharmaceutical composition including a human hyaluronidase PH20 variant having enhanced enzymatic activity and thermal stability and one or more drugs, and a method of treating a disease using the same.

The pharmaceutical composition according to the present invention may preferably be used for subcutaneous administration (subcutaneous injection).

### BACKGROUND ART

Drugs which should be administered in a high dose or in a large amount, especially antibody drugs and the like, are generally administered via intravenous injection, and such injection takes about 90 minutes or longer, an additional preparation procedure should be accompanied for intravenous injection, thus both a patient, doctors and medical staff are inconvenienced, and additional costs are incurred. In contrast, subcutaneous injections have the advantage of enabling immediate administration, but the absorption rate is relatively low compared to intravenous injections, and when the injection amount is 3 to 5 mL or more, it may cause swelling and pain at the injection site, as absorption occurs slowly. As for this reason, subcutaneous injections of protein therapeutic agents is usually limited to solution injection of a small amount of 2 mL or less. However, upon subcutaneous administration (subcutaneous administration or subcutaneous injection) of hyaluronidase along with a therapeutic drug, hyaluronic acid distributed in the extracellular matrix is hydrolyzed by the action of hyaluronidase, and thus the viscosity of the subcutaneous area decreases and the permeability of a substance increases, and therefore, a high dose and a large amount of medicine can easily be delivered into the body.

There are six types of hyaluronidase genes in humans: Hyal1, Hyal2, Hyal3, Hyal4, HyalPS1, and PH20/SPAM1. Hyal1 and Hyal2 are expressed in most tissues, and PH20/SPAM1 (hereinafter, referred to as PH20) is expressed in the sperm cell membrane and the acrosomal membrane. HyalPS1 is not expressed because it is a pseudogene. PH20 is an enzyme (EC 3.2.1.35) that cleaves β-1, 4 bonds between N-acetylglucosamine and glucuronic acid, which are sugars constituting hyaluronic acid. Human hyaluronidase PH20 has an optimal pH of 5.5 but exhibits some activity even at a pH level of 7 to 8, whereas other human hyaluronidases, including Hyal1, have an optimal pH of 3 to 4 and have very weak activity at a pH level of 7 to 8. The pH of subcutaneous areas in a human is about 7.4, which is substantially neutral, and thus, among various types of hyaluronidases, PH20 is widely applied in clinical use. Examples of the clinical use of PH20 include subcutaneous injection of an antibody therapeutic agent, use as an eye relaxant and an anesthetic injection additive in ophthalmic surgery, use in increasing the access of an anticancer therapeutic agent to the tumor cells by hydrolyzing hyaluronic acid in the extracellular matrix of tumor cells, and use in promoting the resorption of body fluids and blood, which are excessively present in tissue, etc.

Meanwhile, currently commercially available PH20 is in a form extracted from the testes of cattle or sheep. Examples thereof include Amphadase® (bovine hyaluronidase) and Vitrase® (sheep hyaluronidase).

Bovine testicular hyaluronidase (BTH) is obtained by removing a signal peptide and 56 amino acids on the C-terminus from bovine wild-type PH20 during post-translational modification of proteins. BTH is also a glycoprotein and has a mannose content of 5% and a glucosamine content of 2.28, based on the total constitution thereof including amino acids (Borders and Raftery, 1968). When animal-derived hyaluronidase is repeatedly administered to the human body at a high dose, a neutralizing antibody can be produced, and other animal-derived biomaterials contained as impurities in addition to PH20 may cause an allergic reaction. In particular, the use of PH20 extracted from cattle is limited due to concern over mad cow disease. In order to overcome these problems, studies on recombinant human PH20 proteins have been conducted.

Recombinant human PH20 proteins have been reported to be expressed in yeast (P. pastoris), DS-2 insect cells, animal cells, and the like (Chen et al., 2016, Hofinger et al., 2007). The recombinant PH20 proteins produced in insect cells and yeast differ from human PH20 in terms of the pattern of N-glycosylation during post-translational modification of proteins.

Among hyaluronidases, the protein structures of Hyal1 (PDB ID: 2PE4) (Chao et al., 2007) and bee venom hyaluronidase (PDB ID: 1FCQ, 1FCU, 1FCV) have been identified. Hyal1 is composed of two domains, a catalytic domain and an EGF-like domain, and the catalytic domain is in the form of (β/α) 8, in which an alpha helix and a beta-strand, which characterize the secondary structure of the protein, are each repeated eight times (Chao et al., 2007). The EGF-like domain is conserved in all variants in which the C-terminus of Hyal1 is spliced differently. The amino acid sequences of Hyal1 and PH20 are 35.1% identical, and the protein tertiary structure of PH20 has not yet been found.

In a structural/functional relationship study of human PH20, the C-terminal region of PH20 was found to be important for protein expression and enzymatic activity, and in particular, it has been reported that termination of the C-terminus with amino acids 477 to 483 is important for enzymatic expression and activity (Frost, 2007). The activity of full-length PH20 (amino acids 1 to 509) or a pH20 variant having a C-terminus truncated at amino acid position 467 or beyond was merely 10% or less of that of a pH20 variant having a C-terminus truncated at one site among positions 477 to 483 (Frost, 2007). Halozyme Therapeutics developed rHuPH20 (amino acids 36 to 482), which is a recombinant protein in the form in which the C-terminus of mature PH20 was truncated at Y482 (Bookbinder et al., 2006; Frost, 2007).

Meanwhile, although research is ongoing to develop various therapeutic drugs in the form of subcutaneous injection formulations using human PH20, the problem of low stability of human PH20 itself still remains unsolved.

Against this technical background, the inventors of the present invention found that human PH20 variants, including one or more amino acid substitutions in an alpha-helix 8 region (S347 to C381) and a linker region (A333 to R346) between alpha-helix 7 and alpha-helix 8 in the amino acid sequence of wild-type hyaluronidase PH20, and in which portion(s) of amino acids located at the N-terminus and/or the C-terminus of PH20 are truncated, had excellent enzymatic activity and thermal stability, and thus filed a patent application therefor (PCT/KR 2019/009215).

The inventors of the present application also found that the PH20 variants according to the present invention may be applied to pharmaceutical compositions or formulations including drugs, e.g., antibody drugs, particularly high-dose anti-HER2 antibodies or immune checkpoint antibodies, and accordingly, pharmaceutical compositions and formulations according to the present invention including a PH20 variant along with a drug such as anti-HER2 antibodies or immune checkpoint antibodies can be used for subcutaneous administration, and the activities of a drug such as antibody drugs and the PH20 variant are very stable and can be maintained for a long time, thus completing the present invention.

### SUMMARY OF THE INVENTION

It is an object of the present invention to provide a novel pharmaceutical composition including a PH20 variant having enhanced enzymatic activity and thermal stability along with a drug, wherein the thermal stability and activity of the drug and the PH20 variant can be maintained for a long time, particularly a pharmaceutical composition that can be used for subcutaneous administration.

It is another object of the present invention to provide a method of treating a disease including administering the pharmaceutical composition according to the present invention to a subject in need of treatment.

In accordance with the present invention, the above and other objects can be accomplished by the provision of a pharmaceutical composition including (a) a drug and (b) a PH20 variant.

The PH20 variant included in the pharmaceutical composition according to the present invention is characterized by including one or more amino acid residue substitutions selected from the group consisting of S343E, M345T, K349E, L353A, L354I, N356E, and I361T in wild-type human PH20 having an amino acid sequence of SEQ ID NO: 1, and is characterized by further including amino acid substitution(s) in one or more regions selected from an alpha-helix 8 region (S347 to C381) and/or a linker region (A333 to R346) between alpha-helix 7 and alpha-helix 8, wherein portion(s) of amino acid residues located at an N-terminus and/or a C-terminus are selectively truncated.

The pharmaceutical composition according to the present invention may further include one or more selected from pharmaceutically acceptable additives, particularly a buffer, a stabilizer, and a surfactant.

The pharmaceutical composition according to the present invention may be used in the form of an injection formulation for subcutaneous administration.

### BRIEF DESCRIPTION OF DRAWINGS

FIG. 1A illustrates size-exclusion chromatography chromatograms of trastuzumab in a stability test under harsh conditions at 45° C., and FIG. 1B illustrates a change in the purity of a trastuzumab monomeric protein according to formulation in a stability test under harsh conditions at 45° C.

FIGS. 2A and 2B illustrate the results of measuring the protein aggregation temperatures of formulations including trastuzumab and a novel PH20 variant HP46.

FIG. 3A is a weak cation exchange (WCX) chromatography chromatogram of trastuzumab in a stability test under harsh conditions at 45° C., FIG. 3B illustrates changes (%) in relative contents of acidic variants in formulations in a stability test under harsh conditions at 45° C., FIG. 3C illustrates changes (%) in relative contents of main peaks for formulations in a stability test under harsh conditions at 45° C., and FIG. 3D illustrates changes (%) in relative contents of basic variants in formulations in a stability test under harsh conditions at 45° C.

FIG. 4 illustrates changes in the purity of a trastuzumab monomeric protein in formulations 5 to 7 in a stability test under harsh conditions at 45° C.

FIG. 5A illustrates changes (%) in relative contents of acidic variants according to formulations 5 to 7 in a stability test under harsh conditions at 45° C., FIG. 5B illustrates changes (%) in relative contents of main peaks according to formulations 5, 6, and 7 in a stability test under harsh conditions at 45° C., and FIG. 5C illustrates changes (%) in relative contents of basic variants according to formulations 5, 6, and 7 in a stability test under harsh conditions at 45° C.

FIG. 6A illustrates the results of measuring the residual enzymatic activity of a Herceptin subcutaneous injection formulation (Herceptin SC), trastuzumab+wild-type PH20 (HW2), and trastuzumab+PH20 variant HP46 on day 0 and day 1 in a stability test under harsh conditions at 40° C., and

FIG. 6B illustrates the results of measuring the residual enzymatic activity of the Herceptin subcutaneous injection formulation, trastuzumab+wild-type PH20 (HW2), and trastuzumab+PH20 variant HP46 on day 0 and day 1 in a stability test under harsh conditions at 45° C.

FIG. 7 illustrates size-exclusion chromatography analysis results of formulations 8 to 10 in a stability test under harsh conditions at 40° C. for 14 days.

FIG. 8A illustrates the results of measuring changes in protein particle size of formulations 8 to 10 using DLS equipment, and FIG. 8B illustrates the results of measuring protein aggregation temperatures.

FIG. 9A illustrates a weak cation exchange (WCX) chromatography chromatogram of formulation 8 in a stability test under harsh conditions at 40° C., FIG. 9B illustrates changes (%) in relative contents of acidic variants in formulations 8 to 10 in a stability test under harsh conditions at 40° C., FIG. 9C illustrates changes (%) in relative contents of main peaks for formulations 8 to 10 in a stability test under harsh conditions at 40° C., and FIG. 9D illustrates changes (%) in relative contents of basic variants in formulations 8 to 10 in a stability test under harsh conditions at 40° C.

FIG. 10 illustrates changes (%) in relative enzymatic activity of formulations 8 to 10 in a stability test under harsh conditions at 40° C.

FIG. 11 illustrates changes in the purity of trastuzumab monomers of formulations 11 to 13 in a stability test under harsh conditions at 40° C.

FIG. 12A illustrates a weak cation exchange (WCX) chromatography chromatogram of formulation 11 in a stability test under harsh conditions at 40° C., FIG. 12B illustrates changes (%) in relative contents of acidic variants in formulations 11 to 13 in a stability test under harsh conditions at 40° C., FIG. 12C illustrates changes (%) in relative contents of main peaks for formulations 11 to 13 in a stability test under harsh conditions at 40° C., and FIG. 12D illustrates changes (%) in relative contents of basic variants in formulations 11 to 13 in a stability test under harsh conditions at 40° C.

FIG. 13 illustrates changes (%) in relative enzymatic activity of formulations 11 to 13 in a stability test under harsh conditions at 40° C.

FIG. 14 illustrates changes in the purity of rituximab monomers of formulations 14 to 16 in a stability test under harsh conditions at 40° C.

FIG. 15 illustrates changes in relative enzymatic activity of formulations 14 to 16 in a stability test under harsh conditions at 40° C.

FIG. 16 illustrates changes in relative enzymatic activity of formulations 17 and 18 in a stability test under harsh conditions at 40° C.

FIG. 17 illustrates size-exclusion chromatography analysis results of formulations 19 to 22 at 40° C.

FIG. 18 illustrates changes in relative enzymatic activity of formulations 19 to 22 in a stability test under harsh conditions at 40° C.

FIG. 19 illustrates changes in enzymatic activity according to changes in pH for recombinant human PH20 and HP46.

FIG. 20 illustrates experimental results of pharmacokinetics of a Herceptin subcutaneous injection product (Herceptin SC) and a Herceptin subcutaneous injection biosimilar candidate (trastuzumab+HP46; Herceptin SC BS) in 9-week-old Sprague-Dawley rats, wherein Herceptin and the Herceptin biosimilar candidate were injected at 18 mg/kg each, and the subcutaneous injection formulation contained 100 units of rHuPH20 and 100 units of HP46 (at pH 5.3).

### DETAILED DESCRIPTION AND PREFERRED EMBODIMENTS OF THE INVENTION

Unless defined otherwise, all technical and scientific terms used herein have the same meanings as those generally understood by a person having ordinary skill in the art to which the invention pertains. In general, the nomenclature used herein is well known and commonly used in the art.

A working example of the present invention relates to a pharmaceutical composition including (a) a drug and (b) a PH20 variant, and the pharmaceutical composition according to the present invention may be used for the prevention or treatment of a disease and is preferably used for subcutaneous administration.

The human PH20 variant included in the pharmaceutical composition according to the present invention is characterized by substitution(s) of portion(s) of amino acid residues in the region corresponding to an alpha-helix region and/or a linker region thereof, preferably an alpha-helix 8 region (S347 to C381) and/or a linker region (A333 to R346) between alpha-helix 7 and alpha-helix 8, more preferably an amino acid region among T341 to N363, and most preferably an amino acid region corresponding to T341 to I361, L342 to I361, S343 to I361, I344 to I361, M345 to I361, or M345 to N363, in the amino acid sequence of wild-type PH20 (having the amino acid sequence of SEQ ID NO: 1), preferably mature wild-type PH20 (having the sequence consisting of L36 to S490 in the amino acid sequence of SEQ ID NO: 1).

In the present invention, “mature wild-type PH20” refers to a protein consisting of amino acid residues L36 to S490 of SEQ ID NO: 1, which lacks M1 to T35, which form a signal peptide, and A491 to L509, which are not related to the substantial function of PH20, in the amino acid sequence of wild-type PH20 having the sequence of SEQ ID NO: 1.

TABLE 1
Amino acid sequence of wild-type PH20
(SEQ ID NO: 1)

MGVLKFKHIFFRSFVKSSGVSQIVFTFLLIPCCLTLNFRAPPVIPNVPFL
WAWNAPSEFCLGKFDEPLDMSLFSFIGSPRINATGQGVTIFYVDRLGYYP
YIDSITGVTVNGGIPQKISLQDHLDKAKKDITFYMPVDNLGMAVIDWEEW
RPTWARNWKPKDVYKNRSIELVQQQNVQLSLTEATEKAKQEFEKAGKDFL
VETIKLGKLLRPNHLWGYYLFPDCYNHHYKKPGYNGSCFNVEIKRNDDLS
WLWNESTALYPSIYLNTQQSPVAATLYVRNRVREAIRVSKIPDAKSPLPV
FAYTRIVFTDQVLKFLSQDELVYTFGETVALGASGIVIWGTLSIMRSMKS
CLLLDNYMETILNPYIINVTLAAKMCSQVLCQEQGVCIRKNWNSSDYLHL
NPDNFAIQLEKGGKFTVRGKPTLEDLEQFSEKEYCSCYSTLSCKEKADVK
DTDAVDVCIADGVCIDAFLKPPMETEEPQIFYNASPSTLSATMEIVSILF
LIISSVASL

Specifically, the PH20 variant or fragment thereof included in the pharmaceutical composition according to the present invention includes one or more mutations, preferably amino acid residue substitutions, selected from the group consisting of S343E, M345T, K349E, L353A, L354I, N356E, and I361T, and most preferably one or more amino acid residue substitutions selected from the group consisting of L354I and N356E, in wild-type PH20 having the sequence of SEQ ID NO: 1.

In the present invention, the term “PH20 variant” is intended to include mutation of portion(s) of amino acid residues, preferably substitution of amino acid residues in the sequence of wild-type human PH20, as well as the deletion of portion(s) of amino acid residues at the N-terminus and/or the C-terminus together with such substitution of amino acid residues, and is used with substantially the same meaning as the expression PH20 variant or a fragment thereof.

The inventors of the present invention have verified novel PH20 variants or fragments thereof with increased enzymatic activity and thermal stability compared to wild-type PH20 can be provided through previous studies, based on experimental results in which, enzymatic activity and a protein aggregation temperature (Tagg.) at a neutral pH increase, when the amino acid sequences of an alpha-helix 8 region and a linker region between alpha-helix 7 and alpha-helix 8 of human PH20 are partially substituted with the amino acid sequences of an alpha-helix 8 region and a linker region between alpha-helix 7 and alpha-helix 8 of Hyal1 with high hydrophilicity.

Accordingly, the PH20 variant included in the pharmaceutical composition according to the present invention includes one or more amino acid residue substitutions selected from the group consisting of S343E, M345T, K349E, L353A, L3541, N356E, and I361T, preferably one or more amino acid residue substitutions selected from the group consisting of L354I and N356E, in the amino acid sequence of wild-type PH20 (having the amino acid sequence of SEQ ID NO: 1), preferably mature wild-type PH20 (having a sequence consisting of L36 to S490 in the amino acid sequence of SEQ ID NO: 1),
in which one or more amino acid residues in the region corresponding to an alpha-helix region and/or a linker region thereof, preferably in an alpha-helix 8 region (S347 to C381) and/or a linker region (A333 to R346) between alpha-helix 7 and alpha-helix 8, more preferably in an amino acid region corresponding to T341 to N363, T341 to I361, L342 to I361, S343 to I361, I344 to I361, M345 to I361, or M345 to N363, are substituted.

Particularly, in the PH20 variant included in the pharmaceutical composition according to the present invention, the alpha-helix 8 region (S347 to C381) and/or the linker region (A333 to R346) between alpha-helix 7 and alpha-helix 8 of wild-type PH20, preferably mature wild-type PH20, may be substituted with portion(s) of amino acid residues in the amino acid sequence of a corresponding region of Hyal1 having the sequence of SEQ ID NO: 51 (see Tables 2 and 3), but not limited thereto.

TABLE 2
Amino acid sequence of wild-type Hyal1
(SEQ ID NO: 51)

MAAHLLPICALFLTLLDMAQGFRGPLLPNRPFTTVWNANTQWCLERHGVD
VDVSVFDVVANPGQTFRGPDMTIFYSSQLGTYPYYTPTGEPVFGGLPQNA
SLIAHLARTFQDILAAIPAPDFSGLAVIDWEAWRPRWAFNWDTKDIYRQR
SRALVQAQHPDWPAPQVEAVAQDQFQGAARAWMAGTLQLGRALRPRGLWG
FYGFPDCYNYDFLSPNYTGQCPSGIRAQNDQLGWLWGQSRALYPSIYMPA
VLEGTGKSQMYVQHRVAEAFRVAVAAGDPNLPVLPYVQIFYDTTNHFLPL
DELEHSLGESAAQGAAGVVLWVSWENTRTKESCQAIKEYMDTTLGPFILN
VTSGALLCSQALCSGHGRCVRRTSHPKALLLLNPASFSIQLTPGGGPLSL
RGALSLEDQAQMAVEFKCRCYPGWQAPWCERKSMW

TABLE 3
Comparison between alpha helixes and amino
acid sequences of PH20 and Hyal1

| | Amino acid sequence of | Amino acid sequence of |
|---|---|---|
| Alpha helix | PH20 | Hyal1 |
| Alpha-helix 1 | P56 to D65 | N39 to G48 |
| Alpha-helix 3 | S119 to M135 | S101 to I117 |
| Alpha-helix 4′ | K161 to N176 | K144 to H159 |
| Alpha-helix 4 | S180 to R211 | P163 to R194 |
| Alpha-helix 5 | F239 to S256 | P222 to S239 |
| Alpha-helix 6 | A274 to D293 | K257 to G277 |
| Alpha-helix 7 | S317 to G332 | P299 to G314 |
| Alpha-helix 8 | S347 to C381 | T329 to C363 |

More specifically, the PH20 variant or fragment thereof included in the pharmaceutical composition according to the present invention includes an amino acid residue substitution of L354I and/or N356E in the amino acid sequence of wild-type PH20, preferably mature wild-type PH20,
and preferably further includes an amino acid residue substitution at one or more positions selected from T341 to N363, particularly at one or more positions selected from the group consisting of T341, L342, S343, I344, M345, S347, M348, K349, L352, L353, D355, E359, I361, and N363, but is not limited thereto, and
more preferably, further includes one or more amino acid residue substitutions selected from the group consisting of T341S, L342W, S343E, I344N, M345T, S347T, M348K, K349E, L352Q, L353A, D355K, E359D, I361T, and N363G, but is not limited thereto.

Preferably, the PH20 variant or fragment thereof included in the pharmaceutical composition according to the present invention may include amino acid residue substitutions of M345T, S347T, M348K, K349E, L352Q, L353A, L354I, D355K, N356E, E359D, and I361T, and may further include one or more amino acid residue substitutions selected from the group consisting of T341S, L342W, S343E, I344N, and N363G, but is not limited thereto.

More preferably, the PH20 variant or fragment thereof included in the pharmaceutical composition according to the present invention may include, but is not limited to, any one substitution selected from the group consisting of the following:
(a) T341S, L342W, S343E, I344N, M345T, S347T, M348K, K349E, L352Q, L353A, L354I, D355K, N356E, E359D, and I361T;
(b) L342W, S343E, I344N, M345T, S347T, M348K, K349E, L352Q, L353A, L354I, D355K, N356E, E359D, and I361T;
(c) M345T, S347T, M348K, K349E, L352Q, L353A, L354I, D355K, N356E, E359D, and I361T;
(d) M345T, S347T, M348K, K349E, L352Q, L353A, L354I, D355K, N356E, E359D, I361T, and N363G;
(e) I344N, M345T, S347T, M348K, K349E, L352Q, L353A, L354I, D355K, N356E, E359D, and I361T; and
(f) S343E, I344N, M345T, S347T, M348K, K349E, L352Q, L353A, L354I, D355K, N356E, E359D, and I361T.

In the present invention, an expression, which is described by one-letter amino acid residue code together with numbers, such as “S347”, means the amino acid residue at the corresponding position in the amino acid sequence of SEQ ID NO: 1.

For example, “S347” means that the amino acid residue at position 347 in the amino acid sequence of SEQ ID NO: 1 is serine. In addition, “S347T” means that serine at position 347 of SEQ ID NO: 1 is substituted with threonine.

The PH20 variant included in the pharmaceutical composition according to the present invention is interpreted as including variants in which the amino acid residue at a specific amino acid residue position is conservatively substituted.

As used herein, the term “conservative substitution” refers to modifications of a PH20 variant that involves the substitution of one or more amino acids with amino acids having similar biochemical properties that do not cause loss of the biological or biochemical function of the corresponding PH20 variant.

A “conservative amino acid substitution” is one in which the amino acid residue is replaced with an amino acid residue having a similar side chain. Families of amino acid residues having similar side chains have been defined and are well known in the art. These families include amino acids with basic side chains (e.g., lysine, arginine, and histidine), amino acids with acidic side chains (e.g., aspartic acid and glutamic acid), amino acids with uncharged polar side chains (e.g., glycine, asparagine, glutamine, serine, threonine, tyrosine, and cysteine), amino acids with nonpolar side chains (e.g., alanine, valine, leucine, isoleucine, proline, phenylalanine, methionine, and tryptophan), amino acids with beta-branched side chains (e.g., threonine, valine, and isoleucine), and amino acids with aromatic side chains (e.g., tyrosine, phenylalanine, tryptophan, and histidine).

It is anticipated that the PH20 variant included in the pharmaceutical composition according to the present invention will still retain the activity thereof despite having conservative amino acid substitutions.

In addition, the PH20 variant or fragment thereof included in the pharmaceutical composition according to the present invention is interpreted as including PH20 variants or fragments thereof having substantially the same function and/or effect as those/that of the PH20 variant or the fragment thereof according to the present invention, and having an amino acid sequence homology of at least 80% or 85%, preferably at least 90%, more preferably at least 95%, and most preferably at least 99% with the PH20 variant or fragment thereof according to the present invention.

The PH20 variants according to the present invention have increased expression levels and an increased protein refolding rate, thereby having increased thermal stability in animal cells compared to mature wild-type PH20. Furthermore, the enzymatic activity of the PH20 variants exceeded or was similar to that of mature wild-type PH20 despite the increase in thermal stability.

Meanwhile, it is known that, when portion(s) of amino acids at the C-terminus, such as S490, of the mature wild-type PH20 are additionally truncated, the enzymatic activity is reduced. However, the PH20 variants according to the present invention exhibited increased thermal stability and increased or similar enzymatic activities compared to the mature wild-type PH20 despite the C-terminus sequences being additionally truncated compared to the mature wild-type PH20. In addition, the PH20 variants maintained enzymatic activities thereof despite the mature wild-type PH20 having sequences that up to five amino acids were truncated from the N-terminus, which indicates that residues starting from P41 of the N-terminus played an important role in protein expression and enzymatic activity.

Accordingly, the PH20 variant included in the pharmaceutical composition according to the present invention includes portion(s) of amino acid residue substitutions in the alpha-helix 8 region (S347 to C381) and/or the linker region (A333 to R346) between alpha-helix 7 and alpha-helix 8 of wild-type PH20, and further includes portion(s) of amino acid residue deletions at the C-terminus and/or the N-terminus, but is not limited thereto.

In one embodiment, the PH20 variant included in the pharmaceutical composition according to the present invention may include portion(s) of amino acid residue deletions at the N-terminus resulting from truncation before an amino acid residue selected from the group consisting of M1 to P42 at the N-terminus of the amino acid sequence of SEQ ID NO: 1, preferably before an amino acid residue L36, N37, F38, R39, A40, P41, or P42, and/or portion(s) of amino acid residue deletions at the C-terminus resulting from truncation after an amino acid residue selected from the group consisting of V455 to W509 at the C-terminus, preferably after an amino acid residue selected from the group consisting of V455 to S490, and most preferably, after an amino acid reside V455, C458, D461, C464, 1465, D466, A467, F468, K470, P471, P472, M473, E474, T475, E476, P478, 1480, Y482, A484, P486, T488, or S490.

The expression “truncation before L36, N37, F38, R39, A40, P41, or P42 at the N-terminus” means, respectively, that amino acid residues from M1 to T35 immediately before L36, amino acid residues from M1 to L36 immediately before N37, amino acid residues from M1 to N37 immediately before F38, amino acid residues from M1 to F38 immediately before R39, amino acid residues from M1 to R39 immediately before A40, amino acid residues from M1 to A40 immediately before P41, or amino acid residues from M1 to P41 immediately before P42 in the amino acid sequence of SEQ ID NO: 1 are removed by truncation. The expression “truncation before M1 at the N-terminus of SEQ ID NO: 1” means that no truncation occurs at the N-terminus.

In addition, the expression “truncation after V455, C458, D461, C464, I465, D466, A467, F468, K470, P471, P472, M473, E474, T475, E476, P478, 1480, Y482, A484, P486, T488, or S490 of the C-terminus” means truncation and removal of the amino acid residues starting with the amino acid residue which immediately follows V455, C458, D461, C464, 1465, D466, A467, F468, K470, P472, M473, E474, T475, E476, P478, 1480, Y482, A484, P486, T488, or S490, respectively, in the sequence of SEQ ID NO: 1. For example, occurrence of truncation after S490 means occurrence of truncation between S490 and A491.

Preferably, the human PH20 variant included in the pharmaceutical composition according to the present invention may have an amino acid sequence selected from the group consisting of the amino acid sequences of SEQ ID NOS: 5 to 50, more preferably the amino acid sequence of SEQ ID NO: 44, but is not limited thereto. In PH20 variants constructed in specific working examples according to the present invention, the sequences of substituted or truncated amino acids are shown in Table 4 below.

TABLE 4

| | Sequence | | |
|---|---|---|---|
| Name | Number | Substitution | Sequence |
| HM1 | 5 | 12 amino acids are | LNFRAPPVIPNVPFLWAWNAPSEFCLGKFDEPLDM |
| | | substituted with | SLFSFIGSPRINATGQGVTIFYVDRLGYYPYIDSI |
| | | M345T, S347T, | TGVIVNGGIPQKISLQDHLDKAKKDITFYMPVDNL |
| | | M348K, K349E, | GMAVIDWEEWRPTWARNWKPKDVYKNRSIELVQQQ |
| | | L352Q, L353A, | NVQLSLTEATEKAKQEFEKAGKDFLVETIKLGKLL |
| | | L354I, D355K, | RPNHLWGYYLFPDCYNHHYKKPGYNGSCFNVEIKR |
| | | N356E, E359D, | NDDLSWLWNESTALYPSIYLNTQQSPVAATLYVRN |
| | | I361T, and N363G. | RVREAIRVSKIPDAKSPLPVFAYTRIVFTDQVLKF |
| | | | LSQDELVYTFGETVALGASGIVIWGILSITRTKES |
| | | | CQAIKEYMDTTLGPYIINVILAAKMCSQVTCQEQG |
| | | | VCIRKNWNSSDYLHLNPDNFAIQLEKGGKFTVRGK |
| | | | PTLEDLEQFSEKEYCSCYSTLSCKEKADVKDIDAV |
| | | | DVCIADGVCIDAFLKPPMETEEPQIFYNASPSTLS |
| HM2 | 6 | 7 amino acids | LNFRAPPVIPNVPFLWAWNAPSEFCLGKEDEPLDM |
| | | are substituted | SLFSFIGSPRINATGQGVTIFYVDRLGYYPYIDSI |
| | | with | TGVIVNGGIPQKISLQDHLDKAKKDITFYMPVDNL |
| | | Y365F, I367L, | GMAVIDWEEWRPTWARNWKPKDVYKNRSIELVQQQ |
| | | L371S, A372G, | NVQLSLTEATEKAKQEFEKAGKDFLVETIKLGKLL |
| | | K374L, M375L, and | RPNHLWGYYLFPDCYNHHYKKPGYNGSCFNVEIKR |
| | | V379A. | NDDLSWLWNESTALYPSIYLNTQQSPVAATLYVRN |
| | | | RVREAIRVSKIPDAKSPLPVFAYTRIVFTDQVLKF |
| | | | LSQDELVYTFGETVALGASGIVIWGILSITRTKES |
| | | | CQAIKEYMDTTLNPFILNVISGALLCSQALCQEQG |
| | | | VCIRKNWNSSDYLHLNPDNFAIQLEKGGKFTVRGK |
| | | | PTLEDLEQFSEKEYCSCYSTLSCKEKADVKDIDAV |
| | | | DVCIADGVCIDAFLKPPMETEEPQIFYNASPSTLS |
| HM3 | 7 | 19 amino acids are | LNFRAPPVIPNVPFLWAWNAPSEFCLGKEDEPLDM |
| | | substituted with | SLFSFIGSPRINATGQGVTIFYVDRLGYYPYIDSI |
| | | M345T, S347T, | TGVIVNGGIPQKISLQDHLDKAKKDITFYMPVDNL |
| | | M348K, K349E, | GMAVIDWEEWRPTWARNWKPKDVYKNRSIELVQQQ |
| | | L352Q, L353A, | NVQLSLTEATEKAKQEFEKAGKDFLVETIKLGKLL |
| | | L354I, D355K, | RPNHLWGYYLFPDCYNHHYKKPGYNGSCFNVEIKR |
| | | N356E, E359D, | NDDLSWLWNESTALYPSIYLNTQQSPVAATLYVRN |
| | | I361T, N363G, | RVREAIRVSKIPDAKSPLPVFAYTRIVFTDQVLKF |
| | | Y365F, I367L, | LSQDELVYTEGETVALGASGIVIWGILSITRTKES |
| | | L371S, A372G, | CQAIKEYMDTTLGPFILNVTSGALLCSQALCQEQG |
| | | K374L, M375L, and | VCIRKNWNSSDYLHLNPDNFAIQLEKGGKFTVRGK |
| | | V379A. | PTLEDLEQFSEKEYCSCYSTLSCKEKADVKDIDAV |
| | | | DVCIADGVCIDAFLKPPMETEEPQIFYNASPSTLS |
| HM4 | 8 | 17 amino acids are | LNFRAPPVIPNVPFLWAWNAPSEFCLGKEDEPLDM |
| | | substituted with | SLFSFIGSPRINATGQGVTIFYVDRLGYYPYIDSI |
| | | G340V, T341S, | TGVIVNGGIPQKISLQDHLDKAKKDITFYMPVDNL |
| | | L342W, S343E, | GMAVIDWEEWRPTWARNWKPKDVYKNRSIELVQQQ |
| | | I344N, M345T, | NVQLSLTEATEKAKQEFEKAGKDFLVETIKLGKLL |
| | | S347T, M348K, | RPNHLWGYYLFPDCYNHHYKKPGYNGSCFNVEIKR |
| | | K349E, L352Q, | NDDLSWLWNESTALYPSIYLNTQQSPVAATLYVRN |
| | | L353A, L354I, | RVREAIRVSKIPDAKSPLPVFAYTRIVFTDQVLKF |
| | | D355K, N356E, | LSQDELVYTEGETVALGASGIVIWVSWENTRTKES |
| | | E359D, I361T, and | CQAIKEYMDTTLGPYIINVTLAAKMCSQVLCQEQG |
| | | N363G. | VCIRKNWNSSDYLHLNPDNFAIQLEKGGKFTVRGK |
| | | | PTLEDLEQFSEKEYCSCYSTLSCKEKADVKDIDAV |
| | | | DVCIADGVCIDAFLKPPMETEEPQIFYNASPSTLS |
| HM6 | 9 | 11 amino acid | LNFRAPPVIPNVPFLWAWNAPSEFCLGKEDEPLDM |
| | | residues are | SLFSFIGSPRINATGQGVTIFYVDRLGYYPYIDSI |
| | | substituted with | TGVIVNGGIPQKISLQDHLDKAKKDITFYMPVDNL |
| | | M345T, S347T, | GMAVIDWEEWRPTWARNWKPKDVYKNRSIELVQQQ |
| | | M348K, K349E, | NVQLSLTEATEKAKQEFEKAGKDFLVETIKLGKLL |
| | | L352Q, L353A, | RPNHLWGYYLFPDCYNHHYKKPGYNGSCFNVEIKR |
| | | L354I, D355K, | NDDLSWLWNESTALYPSIYLNTQQSPVAATLYVRN |
| | | N356E, E359D, and | RVREAIRVSKIPDAKSPLPVFAYTRIVFTDQVLKF |
| | | I361T. | LSQDELVYTEGETVALGASGIVIWGILSITRTKES |
| | | | CQAIKEYMDTTLNPYIINVTLAAKMCSQVLCQEQG |
| | | | VCIRKNWNSSDYLHLNPDNFAIQLEKGGKFTVRGK |
| | | | PTLEDLEQFSEKEYCSCYSTLSCKEKADVKDIDAV |
| | | | DVCIADGVCIDAFLKPPMETEEPQIFYNASPSTLS |
| HM7 | 10 | 16 amino acids are | LNFRAPPVIPNVPFLWAWNAPSEFCLGKEDEPLDM |
| | | substituted with | SLFSFIGSPRINATGQGVTIFYVDRLGYYPYIDSI |
| | | G340V, T341S L342W, | TGVIVNGGIPQKISLQDHLDKAKKDITFYMPVDNL |
| | | 5343E, I344N, | GMAVIDWEEWRPTWARNWKPKDVYKNRSIELVQQQ |
| | | M345T, S347T, | NVQLSLTEATEKAKQEFEKAGKDFLVETIKLGKLL |
| | | M348K, K349E, | RPNHLWGYYLFPDCYNHHYKKPGYNGSCFNVEIKR |
| | | L352Q, L353A, | NDDLSWLWNESTALYPSIYLNTQQSPVAATLYVRN |
| | | L354I, D355K, | RVREAIRVSKIPDAKSPLPVFAYTRIVFTDQVLKF |
| | | N356E, E359D, and | LSQDELVYTEGETVALGASGIVIWVSWENTRTKES |
| | | I361T. | CQAIKEYMDTTLNPYIINVTLAAKMCSQVLCQEQG |
| | | | VCIRKNWNSSDYLHLNPDNFAIQLEKGGKFTVRGK |
| | | | PTLEDLEQFSEKEYCSCYSTLSCKEKADVKDIDAV |
| | | | DVCIADGVCIDAFLKPPMETEEPQIFYNASPSTLS |
| HM8 | 11 | 12 amino acids are | LNFRAPPVIPNVPFLWAWNAPSEFCLGKEDEPLDM |
| | | substituted with | SLFSFIGSPRINATGQGVTIFYVDRLGYYPYIDSI |
| | | I344N, M345T, | TGVIVNGGIPQKISLQDHLDKAKKDITFYMPVDNL |
| | | S347T, M348K, | GMAVIDWEEWRPTWARNWKPKDVYKNRSIELVQQQ |
| | | K349E, L352Q, | NVQLSLTEATEKAKQEFEKAGKDFLVETIKLGKLL |
| | | L353A, L354I, | RPNHLWGYYLFPDCYNHHYKKPGYNGSCFNVEIKR |
| | | D355K, N356E, | NDDLSWLWNESTALYPSIYLNTQQSPVAATLYVRN |
| | | E359D, and I361T. | RVREAIRVSKIPDAKSPLPVFAYTRIVFTDQVLKF |
| | | | LSQDELVYTEGETVALGASGIVIWGILSNTRTKES |
| | | | CQAIKEYMDTTLNPYIINVTLAAKMCSQVLCQEQG |
| | | | VCIRKNWNSSDYLHLNPDNFAIQLEKGGKFTVRGK |
| | | | PTLEDLEQFSEKEYCSCYSTLSCKEKADVKDIDAV |
| | | | DVCIADGVCIDAFLKPPMETEEPQIFYNASPSTLS |
| HM9 | 12 | 13 amino acids are | LNFRAPPVIPNVPFLWAWNAPSEFCLGKEDEPLDM |
| | | substituted with | SLFSFIGSPRINATGQGVTIFYVDRLGYYPYIDSI |
| | | S343E, I344N, | TGVIVNGGIPQKISLQDHLDKAKKDITFYMPVDNL |
| | | M345T, S347T, | GMAVIDWEEWRPTWARNWKPKDVYKNRSIELVQQQ |
| | | M348K, K349E, | NVQLSLTEATEKAKQEFEKAGKDFLVETIKLGKLL |
| | | L352Q, L353A, | RPNHLWGYYLFPDCYNHHYKKPGYNGSCFNVEIKR |
| | | L354I, D355K, | NDDLSWLWNESTALYPSIYLNTQQSPVAATLYVRN |
| | | N356E, E359D, and | RVREAIRVSKIPDAKSPLPVFAYTRIVFTDQVLKF |
| | | I361T. | LSQDELVYTEGETVALGASGIVIWGILENTRTKES |
| | | | CQAIKEYMDTTLNPYIINVTLAAKMCSQVLCQEQG |
| | | | VCIRKNWNSSDYLHLNPDNFAIQLEKGGKFTVRGK |
| | | | PTLEDLEQFSEKEYCSCYSTLSCKEKADVKDIDAV |
| | | | DVCIADGVCIDAFLKPPMETEEPQIFYNASPSTLS |
| HM10 | 13 | 14 amino acid | LNFRAPPVIPNVPFLWAWNAPSEFCLGKEDEPLDM |
| | | residues are | SLFSFIGSPRINATGQGVTIFYVDRLGYYPYIDSI |
| | | substituted with | TGVIVNGGIPQKISLQDHLDKAKKDITFYMPVDNL |
| | | L342W, S343E, | GMAVIDWEEWRPTWARNWKPKDVYKNRSIELVQQQ |
| | | I344N, M345T, | NVQLSLTEATEKAKQEFEKAGKDFLVETIKLGKLL |
| | | S347T, M348K, | RPNHLWGYYLFPDCYNHHYKKPGYNGSCFNVEIKR |
| | | K349E, L352Q, | NDDLSWLWNESTALYPSIYLNTQQSPVAATLYVRN |
| | | L353A, L354I, | RVREAIRVSKIPDAKSPLPVFAYTRIVFTDQVLKF |
| | | D355K, N356E, | LSQDELVYTEGETVALGASGIVIWGTWENTRTKES |
| | | E359D, and I361T. | CQAIKEYMDTTLNPYIINVTLAAKMCSQVLCQEQG |
| | | | VCIRKNWNSSDYLHLNPDNFAIQLEKGGKFTVRGK |
| | | | PTLEDLEQFSEKEYCSCYSTLSCKEKADVKDIDAV |
| | | | DVCIADGVCIDAFLKPPMETEEPQIFYNASPSTLS |
| HM11 | 14 | 13 amino acid | LNFRAPPVIPNVPFLWAWNAPSEFCLGKEDEPLDM |
| | | residues are | SLFSFIGSPRINATGQGVTIFYVDRLGYYPYIDSI |
| | | substituted with | TGVIVNGGIPQKISLQDHLDKAKKDITFYMPVDNL |
| | | M345T, S347T, | GMAVIDWEEWRPTWARNWKPKDVYKNRSIELVQQQ |
| | | M348K, K349E, | NVQLSLTEATEKAKQEFEKAGKDFLVETIKLGKLL |
| | | L352Q, L353A, | RPNHLWGYYLFPDCYNHHYKKPGYNGSCFNVEIKR |
| | | L354I, D355K, | NDDLSWLWNESTALYPSIYLNTQQSPVAATLYVRN |
| | | N356E, E359D, | RVREAIRVSKIPDAKSPLPVFAYTRIVFTDQVLKF |
| | | I361T, Y365F, and | LSQDELVYTFGETVALGASGIVIWGTLSITRTKES |
| | | I367L. | CQAIKEYMDTTLNPFILNVTLAAKMCSQVLCQEQG |
| | | | VCIRKNWNSSDYLHLNPDNFAIQLEKGGKFTVRGK |
| | | | PTLEDLEQFSEKFYCSCYSTLSCKEKADVKDTDAV |
| | | | DVCIADGVCIDAFLKPPMETEEPQIFYNASPSTLS |
| HM12 | 15 | 15 amino acid | LNFRAPPVIPNVPFLWAWNAPSEFCLGKFDEPLDM |
| | | residues are | SLFSFIGSPRINATGQGVTIFYVDRLGYYPYIDSI |
| | | substituted with | TGVTVNGGIPQKISLQDHLDKAKKDITFYMPVDNL |
| | | M345T, S347T, | GMAVIDWEEWRPTWARNWKPKDVYKNRSIELVQQQ |
| | | M348K, K349E, | NVQLSLTEATEKAKQEFEKAGKDFLVETIKLGKLL |
| | | L352Q, L353A, | RPNHLWGYYLFPDCYNHHYKKPGYNGSCFNVEIKR |
| | | L354I, D355K, | NDDLSWLWNESTALYPSIYLNTQQSPVAATLYVRN |
| | | N356E, E359D, | RVREAIRVSKIPDAKSPLPVFAYTRIVFTDQVLKF |
| | | I361T, Y365F, | LSQDELVYTFGETVALGASGIVIWGTLSITRTKES |
| | | I367L, L371S, and | CQAIKEYMDTTLNPFILNVTSGAKMCSQVLCQEQG |
| | | A372G. | VCIRKNWNSSDYLHLNPDNFAIQLEKGGKFTVRGK |
| | | | PTLEDLEQFSEKFYCSCYSTLSCKEKADVKDTDAV |
| | | | DVCIADGVCIDAFLKPPMETEEPQIFYNASPSTLS |
| HM13 | 16 | 11 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | M345T, S347T, | AVIDWEEWRPTWARNWKPKDVYKNRSIELVQQQNV |
| | | M348K, K349E , | QLSLTEATEKAKQEFEKAGKDFLVETIKLGKLLRP |
| | | L352Q, L353A, | NHLWGYYLFPDCYNHHYKKPGYNGSCFNVEIKRND |
| | | L354I, D355K, | DLSWLWNESTALYPSIYLNTQQSPVAATLYVRNRV |
| | | N356E, E359D, and | REAIRVSKIPDAKSPLPVFAYTRIVFTDQVLKFLS |
| | | I361T, and cleavage | QDELVYTFGETVALGASGIVIWGTLSITRTKESCQ |
| | | is performed before | AIKEYMDTTLNPYIINVTLAAKMCSQVTCQEQGVC |
| | | residue F38 at the | IRKNWNSSDYLHLNPDNFAIQLEKGGKFTVRGKPT |
| | | N-terminus. | LEDLEQFSEKFYCSCYSTLSCKEKADVKDTDAVDV |
| | | | CIADGVCIDAFLKPPMETEEPQIFYNASPSTLS |
| HM14 | 17 | 11 amino acids are | LNFRAPPVIPNVPFLWAWNAPSEFCLGKFDEPLDM |
| | | substituted with | SLFSFIGSPRINATGQGVTIFYVDRLGYYPYIDSI |
| | | M345T, S347T, | TGVTVNGGIPQKISLQDHLDKAKKDITFYMPVDNL |
| | | M348K, K349E, | GMAVIDWEEWRPTWARNWKPKDVYKNRSIELVQQQ |
| | | L352Q, L353A, | NVQLSLTEATEKAKQEFEKAGKDFLVETIKLGKLL |
| | | L354I, D355K, | RPNHLWGYYLFPDCYNHHYKKPGYNGSCFNVEIKR |
| | | N356E, E359D, and | NDDLSWLWNESTALYPSIYLNTQQSPVAATLYVRN |
| | | I361T, and cleavage | RVREAIRVSKIPDAKSPLPVFAYTRIVFTDQVLKF |
| | | is performed after | LSQDELVYTFGETVALGASGIVIWGTLSITRTKES |
| | | the carboxyl group | CQAIKEYMDTTLNPYIINVTLAAKMCSQVLCQEQG |
| | | of I465. | VCIRKNWNSSDYLHLNPDNFAIQLEKGGKFTVRGK |
| | | | PTLEDLEQFSEKFYCSCYSTLSCKEKADVKDTDAV |
| | | | DVCIADGVCI |
| HM15 | 18 | 11 amino acids are | LNFRAPPVIPNVPFLWAWNAPSEFCLGKFDEPLDM |
| | | substituted with | SLFSFIGSPRINATGQGVTIFYVDRLGYYPYIDSI |
| | | M345T, S347T, | TGVTVNGGIPQKISLQDHLDKAKKDITFYMPVDNL |
| | | M348K, K349E, | GMAVIDWEEWRPTWARNWKPKDVYKNRSIELVQQQ |
| | | L352Q, L353A, | NVQLSLTEATEKAKQEFEKAGKDFLVETIKLGKLL |
| | | L354I, D355K, | RPNHLWGYYLFPDCYNHHYKKPGYNGSCFNVEIKR |
| | | N356E, E359D, and | NDDLSWLWNESTALYPSIYLNTQQSPVAATLYVRN |
| | | I361T, and cleavage | RVREAIRVSKIPDAKSPLPVFAYTRIVFTDQVLKF |
| | | is performed after | LSQDELVYTFGETVALGASGIVIWGTLSITRTKES |
| | | the carboxyl group | CQAIKEYMDTTLNPYIINVTLAAKMCSQVLCQEQG |
| | | of F468. | VCIRKNWNSSDYLHLNPDNFAIQLEKGGKFTVRGK |
| | | | PTLEDLEQFSEKFYCSCYSTLSCKEKADVKDTDAV |
| | | | DVCIADGVCIDAF |
| HM16 | 19 | 11 amino acids are | LNFRAPPVIPNVPFLWAWNAPSEFCLGKFDEPLDM |
| | | substituted with | SLFSFIGSPRINATGQGVTIFYVDRLGYYPYIDSI |
| | | M345T, S347T, | TGVIVNGGIPQKISLQDHLDKAKKDITFYMPVDNL |
| | | M348K, K349E, | GMAVIDWEEWRPTWARNWKPKDVYKNRSIELVQQQ |
| | | L352Q, L353A, | NVQLSLTEATEKAKQEFEKAGKDFLVETIKLGKLL |
| | | L354I, D355K, | RPNHLWGYYLFPDCYNHHYKKPGYNGSCFNVEIKR |
| | | N356E, E359D, and | NDDLSWLWNESTALYPSIYLNTQQSPVAATLYVRN |
| | | I361T, and cleavage | RVREAIRVSKIPDAKSPLPVFAYTRIVFTDQVLKF |
| | | is performed after | LSQDELVYTFGETVALGASGIVIWGILSITRTKES |
| | | the carboxyl group | CQAIKEYMDTTLNPYIINVTLAAKMCSQVLCQEQG |
| | | of P471. | VCIRKNWNSSDYLHLNPDNFAIQLEKGGKFTVRGK |
| | | | PTLEDLEQFSEKFYCSCYSTLSCKEKADVKDTDAV |
| | | | DVCIADGVCIDAFLKP |
| HM17 | 20 | Amino acids L36 to | FRGPLLPNRPFLWAWNAPSEFCLGKFDEPLDMSLF |
| | | V47 are substituted | SFIGSPRINATGQGVTIFYVDRLGYYPYIDSITGV |
| | | with FRGPLLPNR, and | TVNGGIPQKISLQDHLDKAKKDITFYMPVDNLGMA |
| | | 11 amino acids are | VIDWEEWRPTWARNWKPKDVYKNRSIELVQQQNVQ |
| | | substituted with | LSLTEATEKAKQEFEKAGKDFLVETIKLGKLLRPN |
| | | M345T, S347T, | HLWGYYLFPDCYNHHYKKPGYNGSCFNVEIKRNDD |
| | | M348K, K349E, | LSWLWNESTALYPSIYLNTQQSPVAATLYVRNRVR |
| | | L352Q, L353A, | EAIRVSKIPDAKSPLPVFAYTRIVFTDQVLKFLSQ |
| | | L354I, D355K, | DELVYTFGETVALGASGIVIWGILSITRTKESCQA |
| | | N356E, E359D, and | IKEYMDTTLNPYIINVTLAAKMCSQVLCQEQGVCI |
| | | I361T. | RKNWNSSDYLHLNPDNFAIQLEKGGKFTVRGKPIL |
| | | | EDLEQFSEKFYCSCYSTLSCKEKADVKDTDAVDVC |
| | | | IADGVCIDAFLKPPMETEEPQIFYNASPSTLS |
| HM18 | 21 | Amino acids L36 to | FRGPLLPNRPFTTVWNAPSEFCLGKFDEPLDMSLF |
| | | A52 are substituted | SFIGSPRINATGQGVTIFYVDRLGYYPYIDSITGV |
| | | with | TVNGGIPQKISLQDHLDKAKKDITFYMPVDNLGMA |
| | | FRGPLLPNRPFTTV, and | VIDWEEWRPTWARNWKPKDVYKNRSIELVQQQNVQ |
| | | 11 amino acids are | LSLTEATEKAKQEFEKAGKDFLVETIKLGKLLRPN |
| | | substituted with | HLWGYYLFPDCYNHHYKKPGYNGSCFNVEIKRNDD |
| | | M345T, S347T, | LSWLWNESTALYPSIYLNTQQSPVAATLYVRNRVR |
| | | M348K, K349E | EAIRVSKIPDAKSPLPVFAYTRIVFTDQVLKFLSQ |
| | | L352Q, L353A | DELVYTFGETVALGASGIVIWGILSITRTKESCQA |
| | | L354I D355K | IKEYMDTTLNPYIINVTLAAKMCSQVLCQEQGVCI |
| | | N356E, E359D, and | RKNWNSSDYLHLNPDNFAIQLEKGGKFTVRGKPIL |
| | | I361T. | EDLEQFSEKFYCSCYSTLSCKEKADVKDTDAVDVC |
| | | | IADGVCIDAFLKPPMETEEPQIFYNASPSTLS |
| HM19 | 22 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTFGETVALGASGIVIWGTWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCIDAFLK |
| | | N-terminus and | |
| | | after residue K470 | |
| | | at the C-terminus. | |
| HM20 | 23 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGTWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCIDAF |
| | | N-terminus and | |
| | | after residue F468 | |
| | | at the C-terminus. | |
| HM21 | 24 | 15 amino acid | LNFRAPPVIPNVPFLWAWNAPSEFCLGKEDEPLDM |
| | | residues are | SLFSFIGSPRINATGQGVTIFYVDRLGYYPYIDSI |
| | | substituted with | TGVIVNGGIPQKISLQDHLDKAKKDITFYMPVDNL |
| | | T341S, L342W, | GMAVIDWEEWRPTWARNWKPKDVYKNRSIELVQQQ |
| | | S343E, I344N, | NVQLSLTEATEKAKQEFEKAGKDFLVETIKLGKLL |
| | | M345T, S347T, | RPNHLWGYYLFPDCYNHHYKKPGYNGSCFNVEIKR |
| | | M348K, K349E, | NDDLSWLWNESTALYPSIYLNTQQSPVAATLYVRN |
| | | L352Q, L353A, | RVREAIRVSKIPDAKSPLPVFAYTRIVFTDQVLKF |
| | | L354I, D355K, | LSQDELVYTEGETVALGASGIVIWGSWENTRTKES |
| | | N356E, E359D, and | CQAIKEYMDTTLNPYIINVTLAAKMCSQVLCQEQG |
| | | I361T. | VCIRKNWNSSDYLHLNPDNFAIQLEKGGKFTVRGK |
| | | | PTLEDLEQFSEKEYCSCYSTLSCKEKADVKDIDAV |
| | | | DVCIADGVCIDAFLKPPMETEEPQIFYNASPSTLS |
| HM24 | 25 | 11 amino acid | APPVIPNVPFLWAWNAPSEFCLGKFDEPLDMSLFS |
| | | residues are | FIGSPRINATGQGVTIFYVDRLGYYPYIDSITGVT |
| | | substituted with | VNGGIPQKISLQDHLDKAKKDITFYMPVDNLGMAV |
| | | M345T, S347T | IDWEEWRPTWARNWKPKDVYKNRSIELVQQQNVQL |
| | | M348K, K349E, | SLTEATEKAKQEFEKAGKDFLVETIKLGKLLRPNH |
| | | L352Q, L353A, | LWGYYLFPDCYNHHYKKPGYNGSCFNVEIKRNDDL |
| | | L354I, D355K, | SWLWNESTALYPSIYLNTQQSPVAATLYVRNRVRE |
| | | N356E, E359D, and | AIRVSKIPDAKSPLPVFAYTRIVFTDQVLKFLSQD |
| | | I361T, and cleavage | ELVYTEGETVALGASGIVIWGILSITRTKESCQAI |
| | | is performed before | KEYMDTTLNPYIINVILAAKMCSQVTCQEQGVCIR |
| | | residue A40 at the | KNWNTSTYLHLNPDNFAIQLEKGGKFTVRGKPTLE |
| | | N-terminus. | DLEQFSEKEYCSCYSTLSCKEKADVKDIDAVDVCI |
| | | | ADGVCIDAFLKPPMETEEPQIFYNASPSTLS |
| HM25 | 26 | 11 amino acids are | PVIPNVPFLWAWNAPSEFCLGKFDEPLDMSLFSFI |
| | | substituted with | GSPRINATGQGVTIFYVDRLGYYPYIDSITGVIVN |
| | | M345T, S347T, | GGIPQKISLQDHLDKAKKDITFYMPVDNLGMAVID |
| | | M348K, K349E, | WEEWRPTWARNWKPKDVYKNRSIELVQQQNVQLSL |
| | | L352Q, L353A, | TEATEKAKQEFEKAGKDFLVETIKLGKLLRPNHLW |
| | | L354I, D355K, | GYYLFPDCYNHHYKKPGYNGSCFNVEIKRNDDLSW |
| | | N356E, E359D, and | LWNESTALYPSIYLNTQQSPVAATLYVRNRVREAI |
| | | I361T, and cleavage | RVSKIPDAKSPLPVFAYTRIVFTDQVLKFLSQDEL |
| | | is performed before | VYTEGETVALGASGIVIWGILSITRTKESCQAIKE |
| | | residue P42 at the | YMDTTLNPYIINVTLAAKMCSQVLCQEQGVCIRKN |
| | | N-terminus. | WNSSDYLHLNPDNFAIQLEKGGKFTVRGKPTLEDL |
| | | | EQFSEKFYCSCYSTLSCKEKADVKDTDAVDVCIAD |
| | | | GVCIDAFLKPPMETEEPQIFYNASPSTLS |
| HM29 | 27 | 14 amino acid | LNFRAPPVIPNVPFLWAWNAPSEFCLGKEDEPLDM |
| | | residues are | SLFSFIGSPRINATGQGVTIFYVDRLGYYPYIDSI |
| | | substituted with | TGVIVNGGIPQKISLQDHLDKAKKDITFYMPVDNL |
| | | L342W, S343E, | GMAVIDWEEWRPTWARNWKPKDVYKNRSIELVQQQ |
| | | I344N, M345T, | NVQLSLTEATEKAKQEFEKAGKDFLVETIKLGKLL |
| | | S347T, M348K, | RPNHLWGYYLFPDCYNHHYKKPGYNGSCFNVEIKR |
| | | K349E, L352Q, | NDDLSWLWNESTALYPSIYLNTQQSPVAATLYVRN |
| | | L353A, L354I, | RVREAIRVSKIPDAKSPLPVFAYTRIVFTDQVLKF |
| | | D355K, N356E, | LSQDELVYTEGETVALGASGIVIWGTWENTRTKES |
| | | E359D, and I361T, | CQAIKEYMDTTLNPYIINVTLAAKMCSQVLCQEQG |
| | | and cleavage is | VCIRKNWNSSDYLHLNPDNFAIQLEKGGKFTVRGK |
| | | performed before | PTLEDLEQFSEKEYCSCYSTLSCKEKADVKDIDAV |
| | | residue L36 at the | DVCIADGVCIDA |
| | | N-terminus and | |
| | | after residue A467 | |
| | | at the C-terminus. | |
| HM30 | 28 | 14 amino acid | LNFRAPPVIPNVPFLWAWNAPSEFCLGKEDEPLDM |
| | | resides are | SLFSFIGSPRINATGQGVTIFYVDRLGYYPYIDSI |
| | | substituted with | TGVIVNGGIPQKISLQDHLDKAKKDITFYMPVDNL |
| | | L342W, S343E, | GMAVIDWEEWRPTWARNWKPKDVYKNRSIELVQQQ |
| | | I344N, M345T, | NVQLSLTEATEKAKQEFEKAGKDFLVETIKLGKLL |
| | | S347T, M348K, | RPNHLWGYYLFPDCYNHHYKKPGYNGSCFNVEIKR |
| | | K349E, L352Q, | NDDLSWLWNESTALYPSIYLNTQQSPVAATLYVRN |
| | | L353A, L354I, | RVREAIRVSKIPDAKSPLPVFAYTRIVFTDQVLKF |
| | | D355K, N356E, | LSQDELVYTEGETVALGASGIVIWGTWENTRTKES |
| | | E359D, and I361T, | CQAIKEYMDTTLNPYIINVTLAAKMCSQVLCQEQG |
| | | and cleavage is | VCIRKNWNSSDYLHLNPDNFAIQLEKGGKFTVRGK |
| | | performed before | PTLEDLEQFSEKEYCSCYSTLSCKEKADVKDIDAV |
| | | residue L36 at the | DVCIADGVC |
| | | N-terminal and | |
| | | after residue C464 | |
| | | at the C-terminus. | |
| HM31 | 29 | 14 amino acid | LNFRAPPVIPNVPFLWAWNAPSEFCLGKEDEPLDM |
| | | residues are | SLFSFIGSPRINATGQGVTIFYVDRLGYYPYIDSI |
| | | substituted with | TGVIVNGGIPQKISLQDHLDKAKKDITFYMPVDNL |
| | | L342W, S343E, | GMAVIDWEEWRPTWARNWKPKDVYKNRSIELVQQQ |
| | | I344N, M345T, | NVQLSLTEATEKAKQEFEKAGKDFLVETIKLGKLL |
| | | S347T, M348K, | RPNHLWGYYLFPDCYNHHYKKPGYNGSCFNVEIKR |
| | | K349E, L352Q, | NDDLSWLWNESTALYPSIYLNTQQSPVAATLYVRN |
| | | L353A, L354I, | RVREAIRVSKIPDAKSPLPVFAYTRIVFTDQVLKF |
| | | D355K, N356E, | LSQDELVYTEGETVALGASGIVIWGTWENTRTKES |
| | | E359D, and I361T, | CQAIKEYMDTTLNPYIINVTLAAKMCSQVLCQEQG |
| | | and cleavage is | VCIRKNWNSSDYLHLNPDNFAIQLEKGGKFTVRGK |
| | | performed before | PTLEDLEQFSEKEYCSCYSTLSCKEKADVKDIDAV |
| | | residue L36 at the | DVCIAD |
| | | N-terminus and | |
| | | after residue D461 | |
| | | at the C-terminus. | |
| HM32 | 30 | 14 amino acid | LNFRAPPVIPNVPFLWAWNAPSEFCLGKEDEPLDM |
| | | residues are | SLFSFIGSPRINATGQGVTIFYVDRLGYYPYIDSI |
| | | substituted with | TGVIVNGGIPQKISLQDHLDKAKKDITFYMPVDNL |
| | | L342W, S343E, | GMAVIDWEEWRPTWARNWKPKDVYKNRSIELVQQQ |
| | | I344N, M345T, | NVQLSLTEATEKAKQEFEKAGKDFLVETIKLGKLL |
| | | S347T, M348K, | RPNHLWGYYLFPDCYNHHYKKPGYNGSCFNVEIKR |
| | | K349E, L352Q, | NDDLSWLWNESTALYPSIYLNTQQSPVAATLYVRN |
| | | L353A, L354I, | RVREAIRVSKIPDAKSPLPVFAYTRIVFTDQVLKF |
| | | D355K, N356E, | LSQDELVYTEGETVALGASGIVIWGTWENTRTKES |
| | | E359D, and I361T, | CQAIKEYMDTTLNPYIINVTLAAKMCSQVLCQEQG |
| | | and cleavage is | VCIRKNWNSSDYLHLNPDNFAIQLEKGGKFTVRGK |
| | | performed before | PTLEDLEQFSEKEYCSCYSTLSCKEKADVKDIDAV |
| | | residue L36 at the | DVC |
| | | N-terminus and | |
| | | after residue C458 | |
| | | at the C-terminus. | |
| HM33 | 31 | 14 amino acid | LNFRAPPVIPNVPFLWAWNAPSEFCLGKEDEPLDM |
| | | residues are | SLFSFIGSPRINATGQGVTIFYVDRLGYYPYIDSI |
| | | substituted with | TGVIVNGGIPQKISLQDHLDKAKKDITFYMPVDNL |
| | | L342W, S343E, | GMAVIDWEEWRPTWARNWKPKDVYKNRSIELVQQQ |
| | | I344N, M345T, | NVQLSLTEATEKAKQEFEKAGKDFLVETIKLGKLL |
| | | S347T, M348K, | RPNHLWGYYLFPDCYNHHYKKPGYNGSCFNVEIKR |
| | | K349E, L352Q, | NDDLSWLWNESTALYPSIYLNTQQSPVAATLYVRN |
| | | L353A, L354I, | RVREAIRVSKIPDAKSPLPVFAYTRIVFTDQVLKF |
| | | D355K, N356E, | LSQDELVYTEGETVALGASGIVIWGTWENTRTKES |
| | | E359D, and I361T, | CQAIKEYMDTTLNPYIINVTLAAKMCSQVLCQEQG |
| | | and cleavage is | VCIRKNWNSSDYLHLNPDNFAIQLEKGGKFTVRGK |
| | | performed before | PTLEDLEQFSEKEYCSCYSTLSCKEKADVKDIDAV |
| | | residue L36 at the | |
| | | N-terminus and | |
| | | after residue V455 | |
| | | at the C-terminus. | |
| HP34 | 32 | 15 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | T341S, L342W, | AVIDWEEWRPTWARNWKPKDVYKNRSIELVQQQNV |
| | | S343E, I344N, | QLSLTEATEKAKQEFEKAGKDFLVETIKLGKLLRP |
| | | M345T, S347T, | NHLWGYYLFPDCYNHHYKKPGYNGSCFNVEIKRND |
| | | M348K, K349E, | DLSWLWNESTALYPSIYLNTQQSPVAATLYVRNRV |
| | | L352Q, L353A, | REAIRVSKIPDAKSPLPVFAYTRIVFTDQVLKFLS |
| | | L354I, D355K, | QDELVYTEGETVALGASGIVIWGSWENTRTKESCQ |
| | | N356E, E359D, and | AIKEYMDTTLNPYIINVTLAAKMCSQVLCQEQGVC |
| | | I361T, and cleavage | IRKNWNSSDYLHLNPDNFAIQLEKGGKFTVRGKPT |
| | | is performed before | LEDLEQFSEKFYCSCYSTLSCKEKADVKDTDAVDV |
| | | residue F38 at the | CIADGVCIDAFLK |
| | | N-terminus and | |
| | | after residue K470 | |
| | | at the C-terminus. | |
| HM35 | 33 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGTWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCIDAFLKPP |
| | | N-terminus and | |
| | | after residue P472 | |
| | | at the C-terminus. | |
| HM36 | 34 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGTWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCIDAFLKPPM |
| | | N-terminus and | |
| | | after residue M473 | |
| | | at the C-terminus. | |
| HM37 | 35 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGTWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCIDAFLKPPME |
| | | N-terminus and | |
| | | after residue E474 | |
| | | at the C-terminus. | |
| HM38 | 36 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGTWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCIDAFLKPPMET |
| | | N-terminus and | |
| | | after residue T475 | |
| | | at the C-terminus. | |
| HM39 | 37 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGTWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCIDAFLKPPMETE |
| | | N-terminus and | |
| | | after residue E476 | |
| | | at the C-terminus. | |
| HM40 | 38 | 11 amino acid | NFRAPPVIPNVPFLWAWNAPSEFCLGKFDEPLDMS |
| | | residues are | LFSFIGSPRINATGQGVTIFYVDRLGYYPYIDSIT |
| | | substituted with | GVTVNGGIPQKISLQDHLDKAKKDITFYMPVDNLG |
| | | M345T, S347T, | MAVIDWEEWRPTWARNWKPKDVYKNRSIELVQQQN |
| | | M348K, K349E, | VQLSLTEATEKAKQEFEKAGKDFLVETIKLGKLLR |
| | | L352Q, L353A, | PNHLWGYYLFPDCYNHHYKKPGYNGSCFNVEIKRN |
| | | L354I, D355K, | DDLSWLWNESTALYPSIYLNTQQSPVAATLYVRNR |
| | | N356E, E359D, and | VREAIRVSKIPDAKSPLPVFAYTRIVFTDQVLKFL |
| | | I361T, and cleavage | SQDELVYTEGETVALGASGIVIWGILSITRTKESC |
| | | is performed before | QAIKEYMDTTLNPYIINVILAAKMCSQVTCQEQGV |
| | | residue N37 at the | CIRKNWNSSDYLHLNPDNFAIQLEKGGKFTVRGKP |
| | | N-terminus. | TLEDLEQFSEKEYCSCYSTLSCKEKADVKDIDAVD |
| | | | VCIADGVCIDAFLKPPMETEEPQIFYNASPSTLS |
| HM41 | 39 | 11 amino acid | RAPPVIPNVPFLWAWNAPSEFCLGKFDEPLDMSLF |
| | | residues are | SFIGSPRINATGQGVTIFYVDRLGYYPYIDSITGV |
| | | substituted with | TVNGGIPQKISLQDHLDKAKKDITFYMPVDNLGMA |
| | | M345T, S347T, | VIDWEEWRPTWARNWKPKDVYKNRSIELVQQQNVQ |
| | | M348K, K349E, | LSLTEATEKAKQEFEKAGKDFLVETIKLGKLLRPN |
| | | L352Q, L353A, | HLWGYYLFPDCYNHHYKKPGYNGSCFNVEIKRNDD |
| | | L354I, D355K, | LSWLWNESTALYPSIYLNTQQSPVAATLYVRNRVR |
| | | N356E, E359D, and | EAIRVSKIPDAKSPLPVFAYTRIVETDQVLKFLSQ |
| | | I361T, and cleavage | DELVYTEGETVALGASGIVIWGILSITRTKESCQA |
| | | is performed before | IKEYMDTTLNPYIINVTLAAKMCSQVTCQEQGVCI |
| | | residue R39 at the | RKNWNSSDYLHLNPDNFAIQLEKGGKFTVRGKPIL |
| | | N-terminus. | EDLEQFSEKEYCSCYSTLSCKEKADVKDIDAVDVC |
| | | | IADGVCIDAFLKPPMETEEPQIFYNASPSTLS |
| HM42 | 40 | 11 amino acid | PPVIPNVPFLWAWNAPSEFCLGKFDEPLDMSLFSF |
| | | residues are | IGSPRINATGQGVTIFYVDRLGYYPYIDSITGVTV |
| | | substituted with | NGGIPQKISLQDHLDKAKKDITFYMPVDNLGMAVI |
| | | M345T, S347T, | DWEEWRPTWARNWKPKDVYKNRSIELVQQQNVQLS |
| | | M348K, K349E, | LTEATEKAKQEFEKAGKDFLVETIKLGKLLRPNHL |
| | | L352Q, L353A, | WGYYLFPDCYNHHYKKPGYNGSCFNVEIKRNDDLS |
| | | L354I, D355K, | WLWNESTALYPSIYLNTQQSPVAATLYVRNRVREA |
| | | N356E, E359D, and | IRVSKIPDAKSPLPVFAYTRIVFTDQVLKFLSQDE |
| | | I361T, and cleavage | LVYTEGETVALGASGIVIWGILSITRTKESCQAIK |
| | | is performed before | EYMDTTLNPYIINVTLAAKMCSQVTCQEQGVCIRK |
| | | residue P41 at the | NWNTSTYLHLNPDNFAIQLEKGGKFTVRGKPTLED |
| | | N-terminus. | LEQFSEKEYCSCYSTLSCKEKADVKDIDAVDVCIA |
| | | | DGVCIDAFLKPPMETEEPQIFYNASPSTLS |
| HM43 | 41 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGSWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCI |
| | | N-terminus and | |
| | | after residue 1465 | |
| | | at the C-terminus. | |
| HM44 | 42 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGSWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCID |
| | | N-terminus and | |
| | | after residue D466 | |
| | | at the C-terminus. | |
| HM45 | 43 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGSWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCIDA |
| | | N-terminus and | |
| | | after residue A467 | |
| | | at the C-terminus. | |
| HP46 | 44 | 15 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | T341S, L342W, | AVIDWEEWRPTWARNWKPKDVYKNRSIELVQQQNV |
| | | S343E, I344N, | QLSLTEATEKAKQEFEKAGKDFLVETIKLGKLLRP |
| | | M345T, S347T, | NHLWGYYLFPDCYNHHYKKPGYNGSCFNVEIKRND |
| | | M348K, K349E, | DLSWLWNESTALYPSIYLNTQQSPVAATLYVRNRV |
| | | L352Q, L353A, | REAIRVSKIPDAKSPLPVFAYTRIVFTDQVLKFLS |
| | | L354I, D355K, | QDELVYTEGETVALGASGIVIWGSWENTRTKESCQ |
| | | N356E, E359D, and | AIKEYMDTTLNPYIINVTLAAKMCSQVLCQEQGVC |
| | | I361T, and cleavage | IRKNWNSSDYLHLNPDNFAIQLEKGGKFTVRGKPT |
| | | is performed before | LEDLEQFSEKFYCSCYSTLSCKEKADVKDTDAVDV |
| | | residue F38 at the | CIADGVCIDAF |
| | | N-terminus and | |
| | | after residue F468 | |
| | | at the C-terminus. | |
| HM47 | 45 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGTWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCIDAFLKPPMETEEP |
| | | N-terminus and | |
| | | after residue P478 | |
| | | at the C-terminus. | |
| HM48 | 46 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGTWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCIDAFLKPPMETEEPQI |
| | | N-terminal and | |
| | | after residue I480 | |
| | | at the C-terminus. | |
| HM49 | 47 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGTWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCIDAFLKPPMETEEPQIFY |
| | | N-terminus and | |
| | | after residue Y482 | |
| | | at the C-terminus. | |
| HM50 | 48 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGTWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCIDAFLKPPMETEEPQIFYNA |
| | | N-terminus and | |
| | | after residue A484 | |
| | | at the C-terminus. | |
| HM51 | 49 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGTWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCIDAFLKPPMETEEPQIFYNASP |
| | | N-terminus and | |
| | | after residue P486 | |
| | | at the C-terminus. | |
| HM52 | 50 | 14 amino acid | FRAPPVIPNVPFLWAWNAPSEFCLGKFDEPLDMSL |
| | | residues are | FSFIGSPRINATGQGVTIFYVDRLGYYPYIDSITG |
| | | substituted with | VTVNGGIPQKISLQDHLDKAKKDITFYMPVDNLGM |
| | | L342W, S343E, | AVIDWEEWRPTWARNWKPKDVYKNRSIELVQQQNV |
| | | I344N, M345T, | QLSLTEATEKAKQEFEKAGKDFLVETIKLGKLLRP |
| | | S347T, M348K, | NHLWGYYLFPDCYNHHYKKPGYNGSCFNVEIKRND |
| | | K349E, L352Q, | DLSWLWNESTALYPSIYLNTQQSPVAATLYVRNRV |
| | | L353A, L354I, | REAIRVSKIPDAKSPLPVFAYTRIVFTDQVLKFLS |
| | | D355K, N356E, | QDELVYTEGETVALGASGIVIWGTWENTRTKESCQ |
| | | E359D, and I361T, | AIKEYMDTTLNPYIINVTLAAKMCSQVLCQEQGVC |
| | | and cleavage is | IRKNWNSSDYLHLNPDNFAIQLEKGGKFTVRGKPT |
| | | performed before | LEDLEQFSEKFYCSCYSTLSCKEKADVKDTDAVDV |
| | | residue F38 at the | CIADGVCIDAFLKPPMETEEPQIFYNASPST |
| | | N-terminus and | |
| | | after residue T488 | |
| | | at the C-terminus. | |

Meanwhile, previous studies reported that the enzymatic activity of wild-type human PH20 changes depending on the truncation positions of amino acid residues located at the C-terminus. In the present invention, however, a specific alpha helix forming the secondary structure of human PH20 was substituted with the alpha helix of other human hyaluronidase, thereby constructing human PH20 variants having higher stability than wild-type human PH20, and in these variants, the interaction between the substituted alpha-helix domain and other secondary structures of PH20 shows a pattern different from that of wild-type PH20, so that the variants are characterized by having a certain level of enzymatic activity or higher, regardless of the truncation position at the C-terminus.

In addition, in the present invention, attempts were made to increase the expression of a recombinant PH20 protein by using the signal peptide of other proteins exhibiting high protein expression levels in animal cells, instead of the original signal peptide of human PH20.

Therefore, in another aspect, the PH20 variant included in the pharmaceutical composition according to the present invention is characterized by including, at the N-terminus thereof, a signal peptide derived from human hyaluronidase-1 (Hyal1), a human growth hormone, or human serum albumin, instead of a signal peptide of wild-type PH20 of M1 to T35, and preferably including, as shown in Table 5, a human-growth-hormone-derived signal peptide having the amino acid sequence of MATGSRTSLLLAFGLLCLPWLQEGSA according to SEQ ID NO: 2, a human serum albumin-derived signal peptide having the amino acid sequence of MKWVTFISLLFLESSAYS according to SEQ ID NO: 3, or a human-Hyal1-derived signal peptide having the amino acid sequence of MAAHLLPICALFLTLLDMAQG according to SEQ ID NO: 4, but is not limited thereto.

TABLE 5
Amino acid sequence of signal peptide of human
growth hormone, human serum albumin, or human Hyal1

| Origin of signal | | SEQ |
|---|---|---|
| peptide | Amino acid sequence | ID NO: |
| Human growth | MATGSRTSLLLAFGLLCLPWLQEGSA | 2 |
| hormone | | |
| Human serum | MKWVTFISLLFLFSSAYS | 3 |
| albumin | | |
| Human Hyal1 | MAAHLLPICALFLTLLDMAQG | 4 |

Among the PH20 variants included in the pharmaceutical composition according to the present invention, a variant having a 6×His-tag attached to the C-terminus was named HM, and a variant without the 6×His-tag was named HP. In addition, mature wild-type PH20 (L36 to S490) with a 6×His-tag attached to the C-terminus thereof was named WT, and mature wild-type PH20 (L36 to Y482) without the 6×His-tag and in which the C-terminus is truncated after Y482 was named HW2.

HP46 (SEQ ID NO: 44) is a human PH20 variant obtained by modeling a protein structure using Hyal1 (PDB ID: 2PE4) (Chao et al., 2007), which is human hyaluronidase, with a known protein tertiary structure, and then substituting the amino acid sequence of amino acids of alpha-helix 8 and the linker region between alpha-helix 7 and alpha-helix 8 of human PH20 with the amino acid sequence of Hyal1, and subjecting the N-terminus to truncation at F38 and subjecting the C-terminus to truncation after F468. In particular, alpha-helix 8 is located outside the protein tertiary structure of PH20 and has less interaction with neighboring alpha helices or beta-strands than other alpha helices. In general, enzymatic activity and thermal stability have a trade-off relationship therebetween, and thus the higher the thermal stability of a protein, the lower the enzymatic activity, whereas, when enzymatic activity increases due to an improvement in the flexibility of the protein structure, the thermal stability tends to be reduced. However, the specific activity of HP46, measured by Turbidimetric assay under conditions at pH 7.0, was about 46 units/μg, which was evaluated to be about two times that of wild-type PH20, which was about 23 units/μg.

The thermal stability of a protein may be evaluated based on a melting temperature (Tm), at which 50% of the protein tertiary structure is denatured, and on an aggregation temperature (Tagg), at which aggregation between proteins occurs. In general, the aggregation temperature of a protein tends to be lower than the melting temperature thereof. The alpha-helix 8 of Hyal1 exhibits greater hydrophilicity than the alpha-helix 8 of PH20. The substituted alpha-helix 8 of Hyal1 increases the protein surface hydrophilicity of HP46, thereby causing the effect of delaying aggregation between proteins that occurs due to hydrophobic interactions, and thus the aggregation temperature is 51° C., which is observed to be an increase of 4.5° C. compared to the aggregation temperature of wild-type PH20, which is 46.5° C.

HP46 is a variant in which amino acids in the alpha-helix 8 and the linker region between alpha-helix 7 and alpha-helix 8 of PH20 are substituted, wherein T341 is substituted with serine. When amino acid residue 341 is threonine, the enzyme activity is similar to that of wild-type PH20, but upon substitution with serine, the enzyme activity increases about 2-fold, and it was found that, even in a substrate gel assay, the resultant variant hydrolyzed hyaluronic acid 5 to 6 times more than wild-type PH20. Substrate gel assay involves protein denaturation and refolding processes, which means that the protein tertiary structure refolding and restoration force of HP46 are enhanced compared to wild-type PH20.

The amount of the PH20 variant in the pharmaceutical composition according to the present invention is at least 50 units/mL, preferably in the range of 100 to 20,000 units/mL, more preferably in the range of about 150 to about 18,000 units/mL, still more preferably in the range of 1,000 to 16,000 units/mL, and most preferably in the range of 1,500 to 12,000 units/mL.

Illustrative examples of the drug included in the pharmaceutical composition according to the present invention include, but are not limited to, protein drugs, antibody drugs, small molecules, aptamers, RNAi, antisenses, and cellular therapeutic agents such as chimeric antigen receptor (CAR)-T or CAR-natural killer (NK), and it is possible to use not only currently commercially available drugs but also drugs in clinical trials or under development.

As the drug, a protein drug or an antibody drug may preferably be used.

The “protein drug” included in the pharmaceutical composition according to the present invention is a drug that consists of amino acids, and thus exhibits the effect of treating or preventing a disease through the activity of a protein, is a drug consisting of a protein other than the antibody drug, and may be selected from the group consisting of a cytokine, a therapeutic enzyme, a hormone, a soluble receptor and a fusion protein thereof, insulin or an analogue thereof, bone morphogenetic protein (BMP), erythropoietin (EPO), and a serum-derived protein, but is not limited thereto.

The cytokine included in the pharmaceutical composition according to the present invention may be selected from the group consisting of interferon, interleukin, colony-stimulating factor (CSF), tumor necrosis factor (TNF), and tissue growth factor (TGF), but is not limited thereto.

The illustrative examples of the therapeutic enzyme may include, but are not limited to, β-glucocerebrosidase and agalsidase β.

The soluble receptor included in the pharmaceutical composition according to the present invention means an extracellular domain of the receptor, and the fusion protein thereof means a protein in which the Fc region or the like of an antibody is fused to the soluble receptor. The soluble receptor is a soluble form of a receptor to which a disease-related ligand binds, and illustrative examples thereof include a form in which an Fc region is fused to the TNF-α soluble receptor (e.g., a product containing the ingredient etanercept and forms similar thereto), a form in which an Fc region is fused to the VEGF soluble receptor (a product containing the ingredient aflibercept and forms similar thereto), a form in which an Fc region is fused to CTLA-4 (e.g., a product containing the ingredient abatacept or belatacept and forms similar thereto), a form in which an Fc region is fused to the interleukin 1 soluble receptor (e.g., a product containing the ingredient rilonacept and forms similar thereto), and a form in which an Fc region is fused to the LFA3 soluble receptor (e.g., a product containing the ingredient alefacept and forms similar thereto), but are not limited thereto.

The hormone included in the pharmaceutical composition according to the present invention refers to a hormone injected into the body or an analog thereof for the treatment or prevention of diseases caused by hormone deficiency and the like, and illustrative examples thereof include, but are not limited to, human growth hormone, estrogen, progesterone, etc.

The serum-derived protein included in the pharmaceutical composition according to the present invention is a protein present in plasma, and means both proteins extracted from plasma and produced recombinant proteins, and illustrative examples thereof may include are not limited to, fibrinogen, von Willebrand factor, albumin, thrombin, factor II (FII), factor V (FV), factor VII (FVII), factor VIII (FVIII), factor IX (FIX), factor X (FX), and factor XI (FXI).

The antibody drug included in the pharmaceutical composition according to the present invention may be a monoclonal antibody drug or a polyclonal antibody drug.

The monoclonal antibody drug according to the present invention means a protein containing a monoclonal antibody and a monoclonal antibody fragment that are capable of specifically binding to an antigen related to a specific disease. The monoclonal antibody also includes a bispecific antibody, and the protein containing a monoclonal antibody or fragment thereof conceptually includes an antibody-drug conjugate (ADC).

Examples of the antigen related to a specific disease include 4-1BB, integrin, amyloid beta, angiopoietin (angiopoietin 1 or 2), angiopoietin analog 3, B-cell-activating factor (BAFF), B7-H3, complement 5, CCR4, CD3, CD4, CD6, CD11a, CD19, CD20, CD22, CD30, CD33, CD38, CD52, CD62, CD79b, CD80, CGRP, Claudin-18, complement factor D, CTLA4, DLL3, EGF receptor, hemophilia factor, Fc receptor, FGF23, folate receptor, GD2, GM-CSF, HER2, HER3, interferon receptor, interferon gamma, IgE, IGF-1 receptor, interleukin 1, interleukin 2 receptor, interleukin 4 receptor, interleukin 5, interleukin 5 receptor, interleukin 6, interleukin 6 receptor, interleukin 7, interleukin 12/23, interleukin 13, interleukin 17A, interleukin 17 receptor A, interleukin 31 receptor, interleukin 36 receptor, LAG3, LFA3, NGF, PVSK9, PD-1, PD-L1, RANK-L, SLAMF7, tissue factor, TNF, VEGF, VEGF receptor, and von Willebrand factor (vWF), but are not limited thereto.

The following are examples of proteins, including but not limited to monoclonal antibodies or monoclonal antibody fragments, that target antigens related to a specific disease:
utomilumab as an anti 4-1BB antibody;
natalizumab, etrolizumab, vedolizumab, and bimagrumab as antibodies against integrin;
bapineuzumab, crenezumab, solanezumab, aducanumab, and gantenerumab as antibodies against amyloid beta;
AMG780 against angiopoietin 1 and 2, MEDI 3617 and nesvacumab against angiopoietin 2, and vanucizumab which is a bispecific antibody against angiopoietin 2 and VEGF, as antibodies against angiopoietin;
evinacumab as an antibody against angiopoietin analog 3;
tabalumab, lanalumab, and belimumab as antibodies against B-cell-activating factor (BAFF);
omburtamab as an antibody against B7-H3;
ravulizumab and eculizumab as antibodies against complement 5;
mogamulizumab as an antibody against CCR4;
otelixizumab, teplizumab, and muromonab as antibodies against CD3, tebentafusp as a bispecific antibody against GP100 and CD3, blinatumomab as a bispecific antibody against CD19 and CD3, and REGN1979 as a bispecific antibody against CD20 and CD3;
ibalizumab and zanolimumab as antibodies against CD4;
itolizumab as an antibody against CD6;
efalizumab as an antibody against CD11a;
inebilizumab, tafasitamab, and loncastuximab tesirine which is an ADC, as antibodies against CD19;
ocrelizumab, ublituximab, obinutuzumab, ofatumumab, rituximab, tositumomab, and ibritumomab tiuxetan which is an ADC, as antibodies against CD20;
epratuzumab, inotuzumab ozogamicin which is an ADC, and moxetumomab pasudotox as antibodies against CD22;
brentuximab vedotin as an ADC against CD30;
vadastuximab talirine and gemtuzumab ozogamicin as ADCs against CD33;
daratumumab and isatuximab as antibodies against CD38;
alemtuzumab as an antibody against CD52;
crizanlizumab as an antibody against CD62;
polaruzumab vedotin as an ADC against CD79b;
galiximab as an antibody against CD80;
eptinezumab, fremanezumab, galcanezumab, and erenumab as antibodies against CGRP;
zolbetuximab as an antibody against Claudin-18;
lampalizumab as an antibody against complement factor D;
tremelimumab, zalifrelimab, and ipilimumab as
antibodies against CTLA4;
rovalpituzumab tesirine as an ADC against DLL3;
cetuximab, depatuxizumab, zalutumumab, necitumumab, and panitumumab as antibodies against the EGF receptor;
emicizumab as a bispecific antibody against coagulation factor IX and factor X, which are hemophilia factors;
nipocalimab and rozanolixizumab as antibodies against the Fc receptor;
burosumab as an antibody against FGF23;
farletuzumab as an antibody against the folate receptor and mirvetuximab soravtansine as an ADC against the folate receptor;
dinutuximab and naxitamab as antibodies against GD2;
otilimab as an antibody against GM-CSF;
margetuximab, pertuzumab, and trastuzumab as antibodies against HER2, and trastuzumab deruxtecan, trastuzumab emtansine, and trastuzumab duocarmazine as ADCs against HER2;
patritumab as an antibody against HER3;
anifrolumab as an antibody against interferon receptor;
emapalumab as an antibody against interferon gamma;
ligelizumab and omalizumab as antibodies against IgE;
dalotuzumab, figitumumab, and teprotumumab as antibodies against the IGF-1 receptor;
gebokizumab and canakinumab as antibodies against interleukin 1;
daclizumab and basiliximab as antibodies against the interleukin 2 receptor;
dupilumab as an antibody against the interleukin 4 receptor;
mepolizumab and reslizumab as antibodies against interleukin 5;
benralizumab as an antibody against the interleukin 5 receptor;
clazakizumab, olokizumab, sirukumab, and siltuximab as antibodies against interleukin 6;
sarilumab, satralizumab, tocilizumab, and REGN88 as antibodies against the interleukin 6 receptor;
secukinumab as an antibody against interleukin 7;
ustekinumab and briakinumab as antibodies against interleukin 12/23;
lebrikizumab and tralokinumab as antibodies against interleukin 13;
ixekizumab and bimekizumab as antibodies against interleukin 17A;
brodalumab as an antibody against interleukin 17 receptor A;
brazikumab, guselkumab, risankizumab, tildrakizumab, and mirikizumab as antibodies against interleukin 23;
nemolizumab as an antibody against the interleukin 31 receptor;
spesolimab as an antibody against the interleukin 36 receptor;
relatlimab as an antibody against LAG3;
narsoplimab as an antibody against NASP2;
fasinumab and tanezumab as antibodies against NGF;
alirocumab, evolocumab, and bococizumab as antibodies against PVSK9;
lambrolizumab, balstilimab, camrelizumab, cemiplimab, dostarlimab, prolgolimab, sintilimab, spartalizumab, tislelizumab, pembrolizumab, and nivolumab as antibodies against PD-1;
atezolizumab, avelumab, envafolimab, and durvalumab as antibodies against PD-L1, and bintrafusp alpha as a bispecific antibody against TGF beta and PD-L1;
denosumab as an antibody against RANK-L;
elotuzumab as an antibody against SLAMF7;
concizumab and marstacimab as antibodies against tissue factor;
infliximab, adalimumab, and golimumab as antibodies against TNF, particularly TNFα, certolizumab pegol as an antibody fragment, and ozoralizumab as a bispecific antibody against TNF and albumin;
brolucizumab, ranibizumab, and bevacizumab as antibodies against VEGF, and faricimab as a bispecific antibody against VEGF and Ang2;
ramucirumab as an antibody against the VEGF receptor; and
caplacizumab as an antibody against VWF.

Meanwhile, the overexpression of human epidermal growth factor receptor 2 (HER2), which promotes cell division, is observed in about 20 to 25% of breast cancer patients, and HER2-overexpressing breast cancer progresses quickly, is aggressive, and has a low response to chemotherapy compared to HER2-low-expressed breast cancer, and thus the prognosis thereof is not good. Trastuzumab, which is a monoclonal antibody drug targeting HER2, specifically binds to HER2 on the surfaces of HER2-overexpressing cancer cells to inhibit the signal transduction of cell replication and proliferation, thereby slowing tumor progression. Trastuzumab was approved by the United States Food and Drug Administration (FDA) in 1998 for the treatment of breast cancer in the United States, and in 2003 by the Korea Food and Drug Administration (KFDA) in South Korea. Since then, the efficacy of trastuzumab was also recognized in HER2-overexpressing gastric cancer, and thus has been used as a therapeutic agent for gastric cancer.

A Roche's Herceptin intravenous injection formulation (commercial name: Herceptin) comprises 440 mg of trastuzumab as a main ingredient, and lyophilized trastuzumab is mixed with physiological saline and injected into a vein. On the other hand, a subcutaneous injection formulation of trastuzumab (commercial name: Herceptin SC) is a 5 mL liquid formulation, and contains 600 mg (120 mg/mL) of trastuzumab as a main ingredient, and includes, as additives, 20 mM histidine (pH 5.5), 210 mM trehalose, 10 mM methionine, 0.04% polysorbate 20, and 10,000 units of rHuPH20 (2,000 Units/mL, 0.004%, 40 μg/mL).

The use-by period of Herceptin subcutaneous injection formulations is 21 months. The intravenous injection formulation of trastuzumab is in a lyophilized form and has a use-by period of 30 months, but the subcutaneous injection formulation of trastuzumab is in a liquid state and has a short use-by period of 21 months. For this reason, it can be presumed that the stability of one or more of trastuzumab and recombinant human hyaluronidase PH20 in liquid formulations is limited.

In this context, in the present invention, in view of the characteristics of the PH20 variant according to the present invention, in which, compared to wild-type human hyaluronidase PH20 and recombinant human PH20 available from Halozyme, the PH20 variant not only has increased enzymatic activity, but also has a high measured protein aggregation temperature, thus exhibiting enhanced thermal stability, the use-by period of the subcutaneous injection formulation is set to a long-term period, preferably 21 months or longer.

The content of the antibody drug in the pharmaceutical composition according to the present invention may be in the range of 5 to 500 mg/mL, preferably 20 to 200 mg/mL, more preferably 100 to 150 mg/mL, and most preferably 120±18 mg/mL, for example, about 110 mg/mL, about 120 mg/mL, or about 130 mg/mL.

The polyclonal antibody included in the pharmaceutical composition according to the present invention is preferably a serum antibody, etc., extracted from serum such as immune globulin, but is not limited thereto.

In the case of a small-molecule compound, any drug that requires a rapid effect for prevention or treatment may be used without limitation. For example, morphine-based painkillers may be used (Thomas et al., 2009). In addition, when used as a therapeutic agent for tissue necrosis caused by anticancer drugs, the small-molecule compound may be used alone or in combination with antidote drugs such as Vinca alkaloids and Taxanes (Kreidieh et al., 2016).

The pharmaceutical composition according to the present invention may further include one or more selected from the group consisting of a buffer, a stabilizer, and a surfactant.

The buffer included in the composition according to the present invention may be used without limitation, as long as it enables realization of a pH of 4 to 8, preferably 5 to 7, and the buffer is preferably one or more selected from the group consisting of malate, formate, citrate, acetate, propionate, pyridine, piperazine, cacodylate, succinate, 2-(N-morpholino) ethanesulfonic acid (MES), histidine, Tris, bis-Tris, phosphate, ethanolamine, carbonate, piperazine-N,N′-bis(2-ethanesulfonic acid) (PIPES), imidazole, BIS-TRIS propane, N, N-bis(2-hydroxyethyl)-2-aminoethanesulfonic acid (BES), 3-(N-morpholino) propanesulfonic acid) (MOPS), hydroxyethyl piperazine ethane sulfonic acid (HEPES), pyrophosphate, and triethanolamine, more preferably a histidine buffer, e.g., L-histidine/HCl, but is not limited thereto.

The concentration of the buffer may be in the range of 0.001 to 200 mM, preferably 1 to 50 mM, more preferably 5 to 40 mM, and most preferably 10 to 30 mM.

Stabilizers in the composition according to the present invention may be used without limitation, as long as they are commonly used in the art for the purpose of stabilizing proteins, and preferably, the stabilizers may be, for example, one or more selected from the group consisting of carbohydrates, sugars or hydrates thereof, sugar alcohols or hydrates thereof, and amino acids.

Carbohydrates, sugars, or sugar alcohols used as the stabilizer may be one or more selected from the group consisting of trehalose or hydrates thereof, sucrose, saccharin, glycerol, erythritol, threitol, xylitol, arabitol, ribitol, mannitol, sorbitol, galactitol, fucitol, iditol, inositol, volemitol, isomalt, maltitol, polyglycitol, cyclodextrin, hydroxylpropyl cyclodextrin (Hydroxypropyl Beta-cyclodextrin), and glucose;
and the amino acids may be one or more selected from the group consisting of glutamine, glutamic acid, glycine, lysine, lysilysine, leucine, methionine, valine, serine, selenomethionine, citrulline, arginine, asparagine, aspartic acid, ornithine, isoleucine, taurine, theanine, threonine, tryptophan, tyrosine, phenylalanine, proline, pyrrolysine, histidine, and alanine, but is not limited thereto.

The concentration of the sugars or sugar alcohols used as a stabilizer in the pharmaceutical composition according to the present invention may be in the range of 0.001 to 500 mM, preferably 100 to 300 mM, more preferably 150 to 250 mM, and most preferably 180 to 230 mM, and particularly, may be about 210 mM.

In addition, the concentration of amino acids used as a stabilizer in the pharmaceutical composition according to the present invention may be in the range of 1 to 100 mM, preferably 3 to 30 mM, more preferably 5 to 25 mM, and most preferably 7 to 20 mM, and specifically, may be in the range of about 8 to 15 mM.

The composition according to the invention may further include a surfactant.

Preferably, the surfactant may be a nonionic surfactant such as polyoxyethylene-sorbitan fatty acid ester (polysorbate or Tween), polyethylene-polypropylene glycol, polyoxyethylene-stearate, polyoxyethylene alkyl ethers, e.g., polyoxyethylene monolauryl ether, alkylphenyl polyoxyethylene ether [Triton-X], and a polyoxyethylene-polyoxypropylene copolymer [Poloxamer and Pluronic], and sodium dodecyl sulfate (SDS), but is not limited thereto.

More preferably, polysorbate may be used. The polysorbate may be polysorbate 20 or polysorbate 80, but is not limited thereto.

The concentration of the nonionic surfactant in the pharmaceutical composition according to the present invention may be in the range of 0.0000001% to 0.5% (w/v), preferably 0.000001% to 0.4% (w/v), more preferably 0.00001% to 0.3% (w/v), and most preferably 0.001% to 0.2% (w/v).

In one specific working example, the pharmaceutical composition according to the present invention may include 50 to 350 mg/mL of an antibody, for example, an anti-HER2 antibody or an immune checkpoint antibody, histidine buffer providing a pH of 5.5±2.0, 10 to 400 mM α,α-trehalose, 1 to 50 mM methionine, and 0.0000001% to 0.5% (w/v) of polysorbate.

In a more specific working example, the pharmaceutical composition according to the present invention may include 120 mg/mL of an anti-HER2 antibody or an immune checkpoint antibody, 20 mM histidine buffer that provides a pH of 5.5±2.0, 210 mM α,α-trehalose, 10 mM methionine, and 2,000 units/mL of a PH20 variant, and may further include 0.005% to 0.1% (w/v) polysorbate.

The pharmaceutical composition according to the present invention may be administered via intravenous injection, subcutaneous injection, intramuscular injection, intraperitoneal injection, endothelial administration, topical administration, intranasal administration, intrapulmonary administration, intrarectal administration, and the like, and subcutaneous administration is preferably performed via subcutaneous injection, and it is more preferable to use the pharmaceutical composition as an injection formulation for subcutaneous injection administration.

Therefore, another embodiment of the present invention provides a formulation including the pharmaceutical composition according to the present invention, preferably an injection formulation for subcutaneous administration.

The injection formulation for subcutaneous administration may be provided in a ready-to-inject form without an additional dilution process, and may be provided after being contained in a pre-filled syringe, a glass ampoule, or a plastic container.

The present invention also relates to a method of treating a disease using the pharmaceutical composition or formulation according to the present invention.

The disease that can be treated using the pharmaceutical composition or formulation according to the present invention is not particularly limited, and there is no limitation thereto, as long as it is a disease that can be treated with a drug used in combination with the PH20 variant according to the present invention.

The disease that can be treated using the pharmaceutical composition or formulation according to the present invention may be cancer or an autoimmune disease, but is not limited thereto.

The cancer or carcinoma treatable with the pharmaceutical composition or formulation according to the present invention is not particularly limited, and includes both solid cancers and blood cancers. Examples of such cancers may be selected from the group consisting of skin cancer such as melanoma, liver cancer, hepatocellular carcinoma, gastric cancer, breast cancer, lung cancer, ovarian cancer, bronchial cancer, nasopharyngeal cancer, laryngeal cancer, pancreatic cancer, bladder cancer, colorectal cancer, colon cancer, cervical cancer, brain cancer, prostate cancer, bone cancer, thyroid cancer, parathyroid cancer, kidney cancer, esophageal cancer, biliary tract cancer, testicular cancer, rectal cancer, head and neck cancer, spinal cancer, ureteral cancer, osteosarcoma, neuroblastoma, fibrosarcoma, rhabdomyosarcoma, astrocytoma, neuroblastoma, and glioma, but are not limited thereto. Preferably, the cancer that can be treated using the pharmaceutical composition or formulation of the present invention may be selected from the group consisting of gastric cancer, colorectal cancer, breast cancer, lung cancer, and kidney cancer, but is not limited thereto.

Autoimmune diseases treatable with the pharmaceutical composition or formulation according to the present invention may be selected from the group consisting of rheumatoid arthritis, asthma, psoriasis, multiple sclerosis, allergic rhinitis, Crohn's disease, ulcerative colitis, systemic erythematous lupus, type I diabetes, inflammatory bowel disease (IBD), and atopic dermatitis, but are not limited thereto.

The present invention also provides a method of treating a disease characterized by administering the pharmaceutical composition or formulation according to the present invention to a subject in need of treatment, and the present invention further provides the use of the pharmaceutical composition or formulation according to the present invention for preparing a medicament for the treatment of a disease.

Unless otherwise defined herein, the technical terms and scientific terms used in the present invention have meanings generally understood by those of ordinary skill in the art. In addition, repeated descriptions of the same technical configuration and operation as those of the related art will be omitted.

Hereinafter, the present invention will be described in further detail with reference to the working examples. These working examples are provided for illustrative purposes only, and it will be obvious to those of ordinary skill in the art that these working examples should not be construed as limiting the scope of the present invention.

### WORKING EXAMPLES

### Working Example 1. Formulation Development

Four types of trastuzumab subcutaneous injection formulations were prepared as shown in Table 6. Formulations 1 to 4 commonly contain 120 mg/mL of trastuzumab, 20 mM histidine/histidine-HCl (pH 5.5), 210 mM trehalose, 10 mM methionine, and a PH20 variant. The difference among formulations 1 to 4 is the concentration of a nonionic surfactant, wherein formulation 1:0% polysorbate 20, formulation 2:0.005% polysorbate 20, formulation 3:0.04% polysorbate 20, and formulation 4:0.1% polysorbate 20.

TABLE 6
Composition of formulations

| | Formulation | Formulation | Formulation | Formulation |
|---|---|---|---|---|
| | 1 | 2 | 3 | 4 |
| Antibody | Trastuzumab (120 mg/mL) | | | |
| Buffer | 20 mM histidine/histidine-HCl | | | |
| Stabilizer 1 | 210 mM trehalose | | | |
| Stabilizer 2 | 10 mM methionine | | | |
| Polysorbate 20 | 0% | 0.005% | 0.04% | 0.1% |

| | |
|---|---|
| Hyaluronidase | HP46 of SEQ ID NO: 44 (2,000 units/mL) |

### Working Example 2. Measurement Using Spectrophotometer

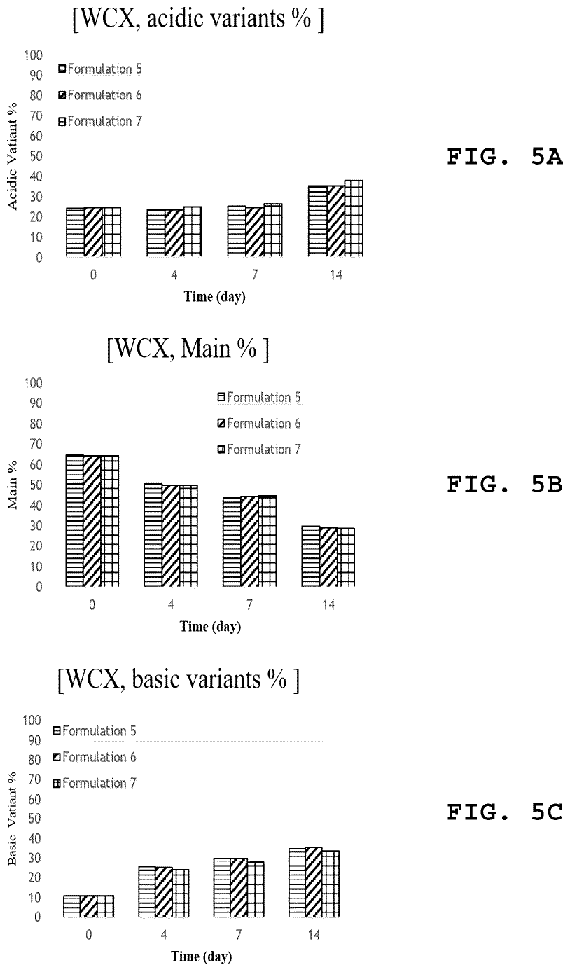

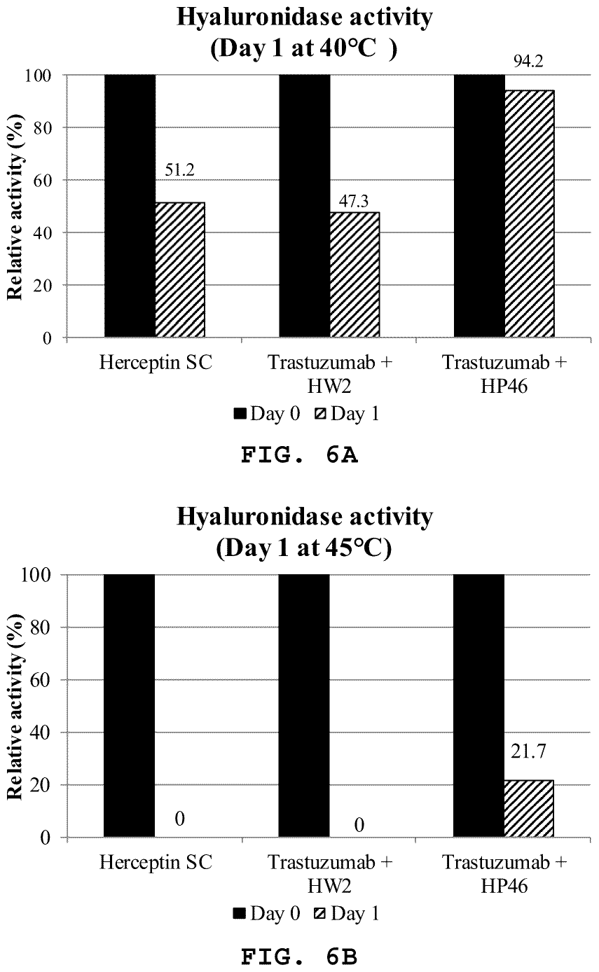

Formulations 1 to 4 were left alone for 14 days at 45° C., and changes in protein concentration were analyzed using a spectrophotometer manufactured by Beckman. Each sample was diluted with distilled water so that the concentration of the sample was 0.4 mg/mL, and then absorbance at 280 nm of the protein was measured using a spectrophotometer. In a stability test under harsh conditions at 45° C. for 14 days, there was no significant change in protein concentration of formulations 1 to 4. However, the activity of hyaluronidase was rapidly reduced at 45° C., and thus, in the present working example, enzymatic activity was not measured (see FIGS. 6A and 6B).

### Working Example 3. Investigation of Monomer Ratio of Trastuzumab in Each Formulation Using Size-Exclusion Chromatography

For size-exclusion chromatography analysis, an HPLC system available from Shimadzu Prominence and a TSK-gel G3000SWXL (7.8×300 mm, 5 μm) and a TSK guard column (6.0×4.0 mm, 7 μm) were used. As a mobile phase, 0.2 M potassium phosphate (pH 6.2) containing 0.25 M potassium chloride was used. Analysis was performed for 35 minutes by applying an isocratic separation mode at a flow rate of 0.5 mL/min. The sample was diluted with an analytical solvent so that the final concentration was 10 mg/mL, and after injecting 20 μL into the HPLC column, absorbance at 280 nm of the column eluate was recorded. The monomer ratio of trastuzumab in the HPLC chromatogram was calculated and graphed.

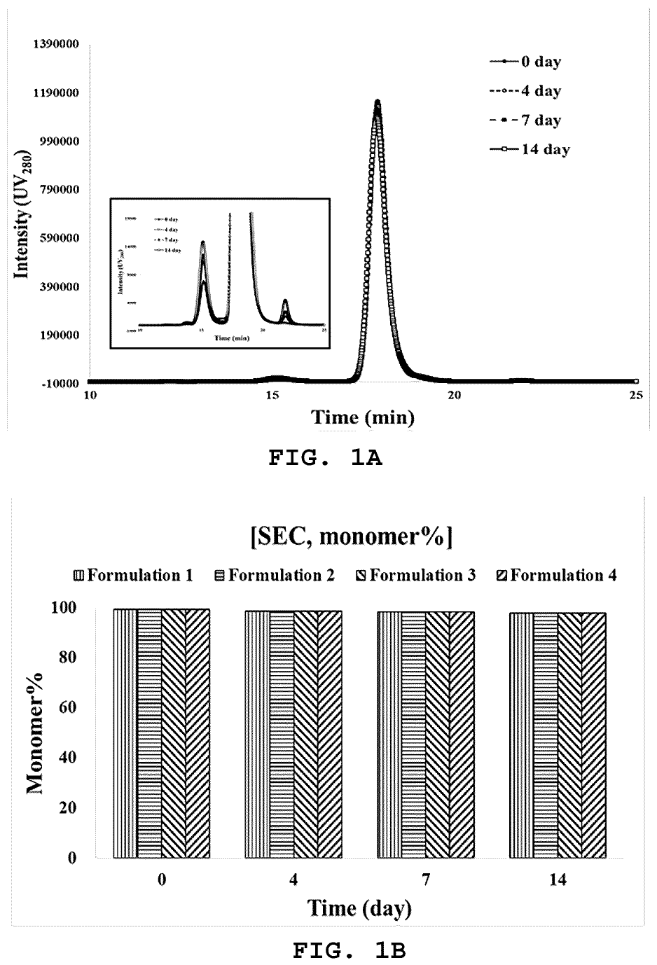

When size-exclusion chromatography analysis was performed in a stability test under harsh conditions at 45° C. for 14 days, formulations 1 to 4 showed similar change patterns. The major changes were increases in high-molecular-weight (HMW) and low-molecular-weight (LMW) degradation products and a decrease in monomer content (about 1.5%), and there was no significant difference according to formulation. In conclusion, as a result of performing size-exclusion chromatography analysis in a stability test under harsh conditions at 45° C., there was no significant difference in stability profile between the formulations according to the concentration of polysorbate 20 (0 to 0.1% (w/v)) (see FIGS. 1A and 1B).

### Working Example 4. Measurement of Protein Aggregation Temperature of Formulations Containing Trastuzumab and HP46

Dynamic light scattering (DLS) is used to analyze the denaturation properties of proteins attributable to heat. In the present experiment, a change in the size of a protein molecule according to the temperature change was measured and used for the purpose of calculating the protein aggregation temperature. For DLS analysis, a Zetasizer-nano-ZS instrument available from Malvern, and a quartz cuvette (ZEN2112) were used. In the analysis process, the temperature was increased from 25° C. to 85° C. at intervals of 1° C., and the sample was diluted to 1 mg/mL using each formulation buffer, and then 150 μL of the sample was added to the cuvette for analysis.

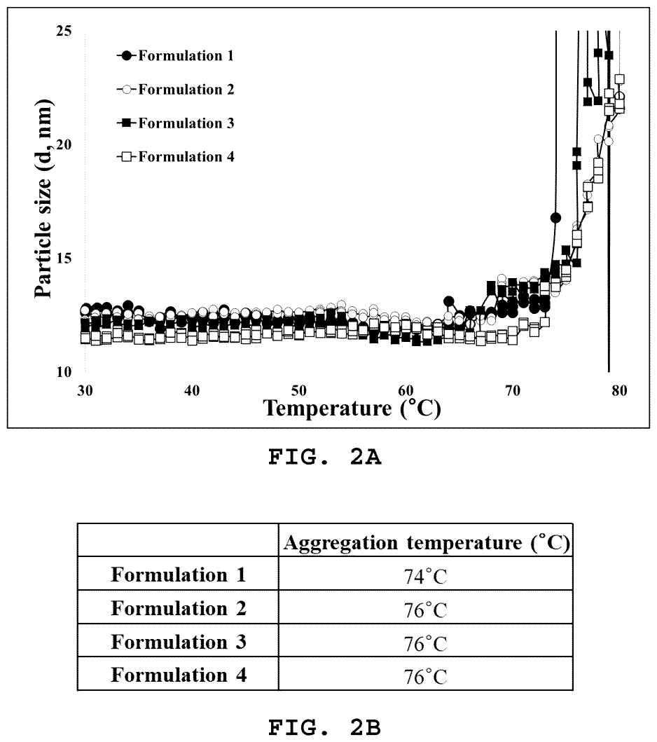

The aggregation temperature in formulation 1, not containing polysorbate 20, was 74° C., and the aggregation temperature in formulations 2 to 4 was 76° C. (see FIGS. 2A and 2B)).

### Working Example 5. WCX Chromatography Measurements for Formulations Containing Trastuzumab and HP46

For WCX chromatography analysis, an HPLC system available from Shimadzu Prominence, and as columns, a TSKgel CM-STAT column (4.6×100 mm, 7 μm), a TSKgel guard gel CMSTAT (3.2 mm I.D.×1.5 cm), and the like were used. Mobile phase A is 10 mM sodium phosphate (pH 7.5) and mobile phase B is 10 mM sodium phosphate (pH 7.2) containing 0.1 M NaCl. Analysis was carried out for 55 minutes with a linear concentration gradient of 0 to 30% mobile phase B at a flow rate of 0.8 mL/min. The sample was diluted with mobile phase A so that the final concentration was 1.0 mg/mL, 80 μL of the sample was injected into HPLC, and then absorbance of a column eluate at 280 nm was recorded. The monomer ratio of trastuzumab in the HPLC chromatogram was calculated and graphed.

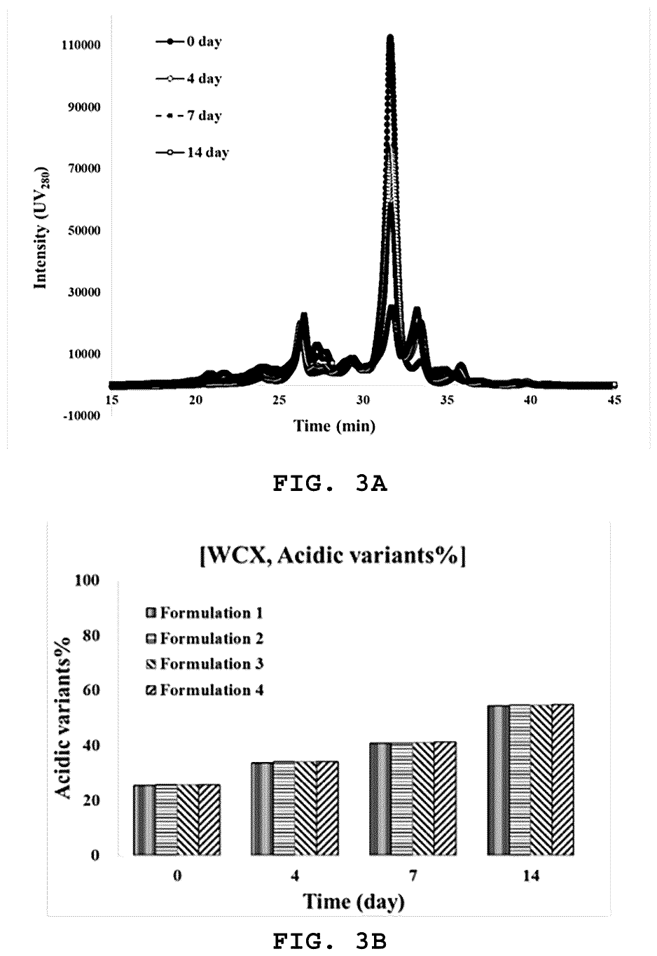

Formulations 1 to 4 showed similar change patterns when WCX analysis was performed in a stability test under harsh conditions at 45° C. for 14 days. Specific changes include an increase in the relative content of acidic variants (approximately 30% change for 14 days), a decrease in the relative content of main peaks (approximately 44% change for 14 days), and an increase in the relative content of basic variants (approximately 15% change for 14 days), and there was no significant difference according to formulation. In conclusion, in the WCX analysis in a stability test under harsh conditions at 45° C., protein stability according to polysorbate 20 (0 to 0.1%) was similar (see FIGS. 3A-3D).

### Working Example 6. Formulation Development

Three types of trastuzumab subcutaneous injection formulations were prepared as described in Table 7. Formulations 5 to 7 commonly include 120 mg/mL of trastuzumab, 20 mM histidine/histidine-HCl (pH 5.5), 210 mM trehalose, 10 mM methionine, and HP46. The difference between formulations 5 to 7 is the ingredient of stabilizer 3, comprising formulation 5:0.04% polysorbate 20, formulation 6:50 mm Lys-Lys, and formulation 3: glycine.

TABLE 7
Composition of formulations

| | Formulation | Formulation | Formulation |
|---|---|---|---|
| | 5 | 6 | 7 |
| Antibody | Trastuzumab (120 mg/mL) | | |
| Buffer | 20 mM histidine/histidine-HCl | | |
| Stabilizer 1 | 210 mM trehalose | | |
| Stabilizer 2 | 10 mM methionine | | |
| Stabilizer 3 | 0.04% | 50 mM | 50 mM |
| | polysorbate 20 | Lys-Lys | glycine |

| | |
|---|---|
| Hyaluronidase | HP46 of SEQ ID NO: 44 (2,000 units/mL) |

### Working Example 7. Measurement Using Spectrophotometer

Formulations 5 to 7 were left alone for 14 days at 45° C., and changes in protein concentration were analyzed using a spectrophotometer manufactured by Beckman. Each sample was diluted with distilled water so that the concentration of the sample was 0.4 mg/mL, and then absorbance of the protein at 280 nm was measured using a spectrophotometer. In a stability test under harsh conditions at 45° C. for 14 days, there was no significant change in protein concentration of formulations 5 to 7. However, the activity of hyaluronidase was rapidly reduced at 45° C., and thus, in the present working example, enzymatic activity was not measured (see FIGS. 6A and 6B).

### Working Example 8. Investigation of Monomer Ratio of Trastuzumab in Each Formulation Using Size-Exclusion Chromatography

For size-exclusion chromatography analysis, an HPLC system available from Shimadzu Prominence and as columns, a TSK-gel G3000SWXL (7.8×300 mm, 5 μm) and a TSK guard column (6.0×4.0 mm, 7 μm) were used. As a mobile phase, 0.2 M potassium phosphate (pH 6.2) containing 0.25 M potassium chloride was used. An isocratic separation mode was applied at a flow rate of 0.5 mL/min for 35 minutes. The sample was diluted with an analytical solvent so that the final concentration was 10 mg/mL, and after injecting 20 μL of the sample into the HPLC, absorbance at 280 nm was measured. The monomer ratio of trastuzumab in the HPLC chromatogram was calculated and graphed.

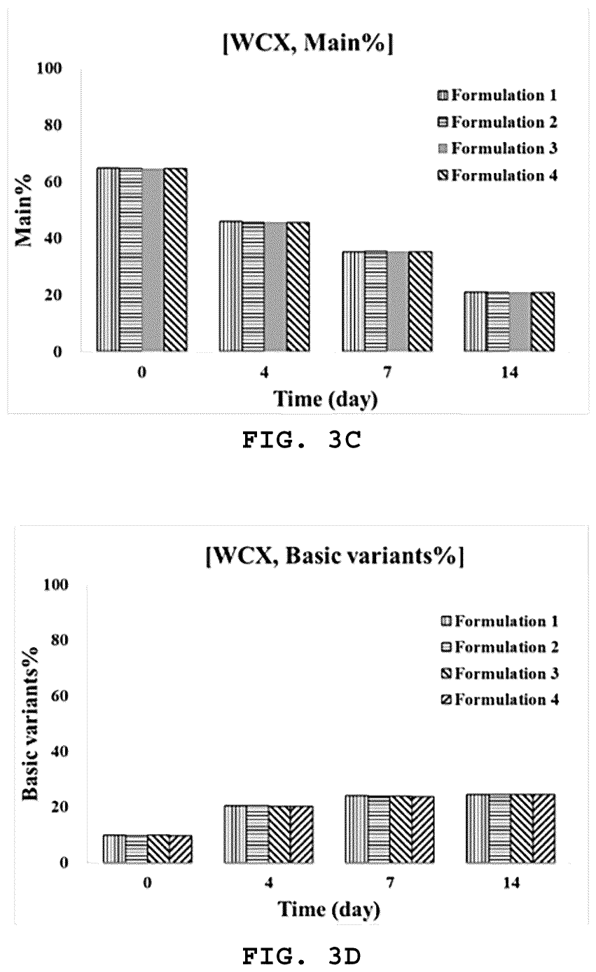

When size-exclusion chromatography analysis was performed in a stability test under harsh conditions at 45° C. for 14 days, formulations 5 to 7 showed similar change patterns. The major changes were increases in high-molecular-weight (HMW) and low-molecular-weight (LMW) impurities and a decrease in monomer content (about 1.5%), and there was no significant difference according to formulation. In conclusion, as a result of performing size-exclusion chromatography analysis in a stability test under harsh conditions at 45° C., similar protein stability was shown in 0.04% polysorbate 20, 50 mM Lys-Lys, and 50 mM glycine formulations (see FIG. 4).

### Working Example 9. WCX Chromatography Analysis of Formulations Containing Trastuzumab and HP46

For WCX chromatography analysis, an HPLC system available from Shimadzu Prominence, and as columns, a TSKgel CM-STAT (4.6×100 mm, 7 μm), a TSKgel guard gel CMSTAT (3.2 mm I.D.×1.5 cm were used. Mobile phase A is 10 mM sodium phosphate (pH 7.5) and mobile phase B is 10 mM sodium phosphate (pH 7.2) containing 0.1 M NaCl. Analysis was performed for 55 minutes by applying a separation mode of a linear concentration gradient of 0 to 30% at a flow rate of 0.8 mL/min. The sample was diluted with mobile phase A so that the final concentration was 1.0 mg/mL, 80 μL of the sample was injected into HPLC, and then absorbance at 280 nm was recorded. The monomer ratio of trastuzumab in the HPLC chromatogram was calculated and graphed.

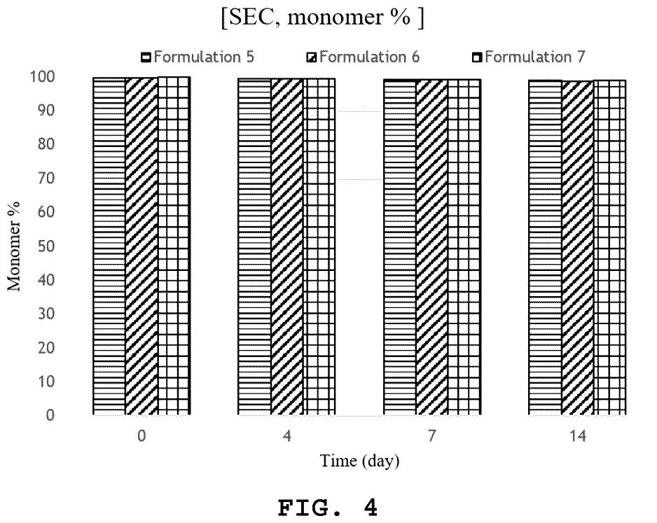

Formulations 5 to 7 showed similar change patterns when WCX analysis was performed in a stability test under harsh conditions at 45° C. for 14 days. Specific changes include an increase in the relative content of acidic variants (approximately 30% change for 14 days), a decrease in the relative content of main peaks (approximately 44% change for 14 days), and an increase in the relative content of basic variants (approximately 15% change for 14 days), and there was no significant difference according to formulation. In conclusion, as a result of performing WCX analysis in a stability test under harsh conditions at 45° C., similar protein stability was shown in 0.04% polysorbate 20, 50 mM Lys-Lys, and 50 mM glycine formulations (see FIGS. 5A-5C).

### Working Example 10. Stability Evaluation of HP46 According to Temperatures of 40° C. And 45° C. In Subcutaneous Injection Formulations of Trastuzumab and HP46

To evaluate the stability of HP46 in subcutaneous injection formulations of trastuzumab, trastuzumab (120 mg/mL) and PH20 (200 units/mL) were mixed. At this time, the buffer used contained 20 mM Histidine (pH 5.5), 210 mM trehalose, 10 mM methionine, and 0.04% polysorbate 20. The enzymatic activity of a control sample was measured on day 0, and the experimental samples were left at 40° C. or 45° C. for one day, and then the enzymatic activity of each sample was measured.

Each of a Herceptin subcutaneous injection formulation, trastuzumab+HW2, and trastuzumab+HP46 was left at 40° C. for one day, and then the activity of hyaluronidase was measured, and as a result, the respective cases exhibited activity of 51%, 47%, and 94%, which indicates that HP46 had a great thermal stability at 40° C. (see FIGS. 6A and 6B). In addition, the Herceptin subcutaneous injection formulation, trastuzumab+HW2, and trastuzumab+HP46 were left at 45° C. for one day, and then the activity of hyaluronidase was measured, and as a result, the enzymatic activity of the Herceptin subcutaneous injection formulation and trastuzumab+HW2 ceased, but the enzymatic activity of trastuzumab+HP46 remained 22% (see FIGS. 6A and 6B).

### Working Example 11. Formulation Development

Three types of trastuzumab subcutaneous injection formulations were prepared as shown in Table 8. Formulations 8 to 10 commonly contain 120 mg/mL of trastuzumab, 20 mM histidine/histidine-HCl (pH 5.5), 210 mM trehalose, 10 mM methionine, and a PH20 variant. The difference among formulations 8 to 10 is the concentration of a nonionic surfactant, wherein formulation 8: 0% polysorbate 20, formulation 9:0.005% polysorbate 20, and formulation 10:0.04% polysorbate 20.

TABLE 8
Composition of formulations

| | Formulation | Formulation | Formulation |
|---|---|---|---|
| | 8 | 9 | 10 |
| Antibody | Trastuzumab (120 mg/mL) | | |
| Polysorbate 20 | 0% | 0.005% | 0.04% |

| | |
|---|---|
| Buffer | 20 mM histidine/histidine-HCl |
| Stabilizer 1 | 210 mM trehalose |
| Stabilizer 2 | 10 mM methionine |
| pH | 5.5 |
| Hyaluronidase | HP46 of SEQ ID NO: 44 (2,000 units/mL) |

### Working Example 12. Measurement Using Spectrophotometer

Formulations 8 to 10 were left alone for 14 days at 40° C., and changes in protein concentration were analyzed using a spectrophotometer manufactured by Beckman. Each sample was diluted with distilled water so that the concentration of the sample was 0.4 mg/mL, and then absorbance at 280 nm of the protein was measured using a spectrophotometer. In a stability test under harsh conditions at 40° C. for 14 days, there was no significant difference in protein concentrations of formulations 8 to 10.

### Working Example 13. Investigation of Monomer Ratio of Trastuzumab in Each Formulation Using Size-Exclusion Chromatography

For size-exclusion chromatography analysis, an HPLC system available from Shimadzu Prominence and as columns, a TSK-gel G3000SWXL (7.8×300 mm, 5 μm) and a TSK guard column (6.0×4.0 mm, 7 μm) were used. As a mobile phase, 0.2 M potassium phosphate (pH 6.2) containing 0.25 M potassium chloride was used. Analysis was performed for 35 minutes by applying an isocratic separation mode at a flow rate of 0.5 mL/min. The sample was diluted with an analytical solvent so that the final concentration was 10 mg/mL, and after injecting 20 μL of the sample into the HPLC column, absorbance at 280 nm was measured. The monomer ratio of trastuzumab in the HPLC chromatogram was calculated and graphed.

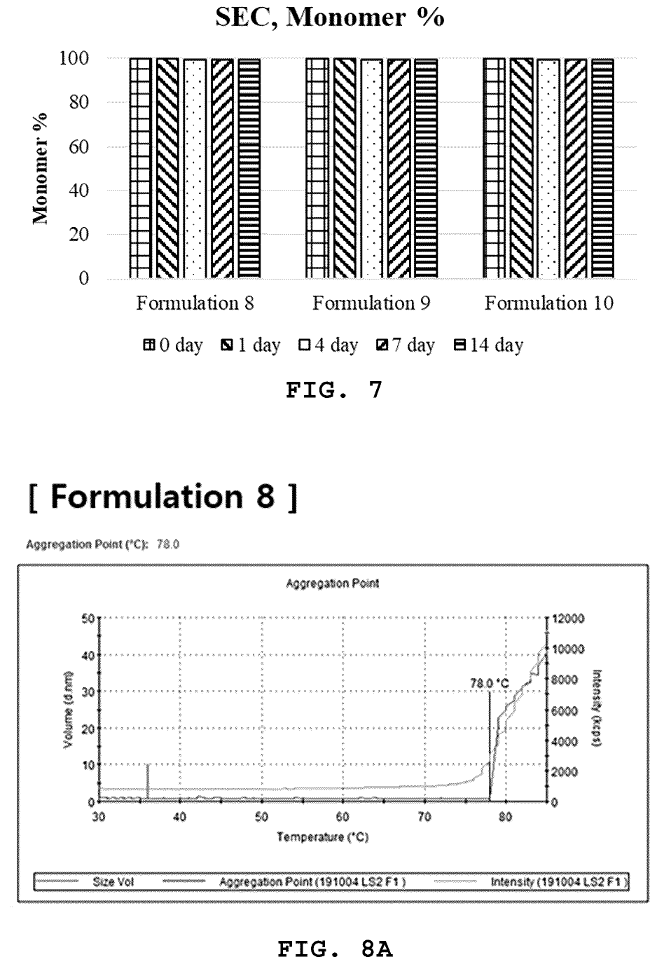

When size-exclusion chromatography analysis was performed in a stability test under harsh conditions at 40° C. for 14 days, formulations 8 to 10 showed similar change patterns. The major changes were increases in high-molecular-weight (HMW) and low-molecular-weight (LMW) degradation products and a decrease in monomer content (about less than 1.0%), and there was no significant difference according to formulation. In conclusion, as a result of performing size-exclusion chromatography analysis in a stability test under harsh conditions at 40° C., there was no significant difference in stability profile between the formulations according to the concentration of polysorbate 20 (0 to 0.04%) (see FIG. 7).

### Working Example 14. Measurement of Protein Aggregation Temperature for Formulations Containing Trastuzumab and HP46

Dynamic light scattering (DLS) is used to analyze the denaturation properties of proteins attributable to heat in the protein drug field. In the present experiment, a change in the size of a protein molecule according to the temperature change was measured and used for the purpose of calculating the protein aggregation temperature. For DLS analysis, a Zetasizer-nano-ZS instrument available from Malvern, and a quartz cuvette (ZEN2112) were used. In the analysis process, the temperature was increased from 25° C. to 85° C. at intervals of 1° C., and the sample was diluted to 1 mg/mL using each formulation buffer, and then 150 μL of the sample was added to the cuvette for analysis.

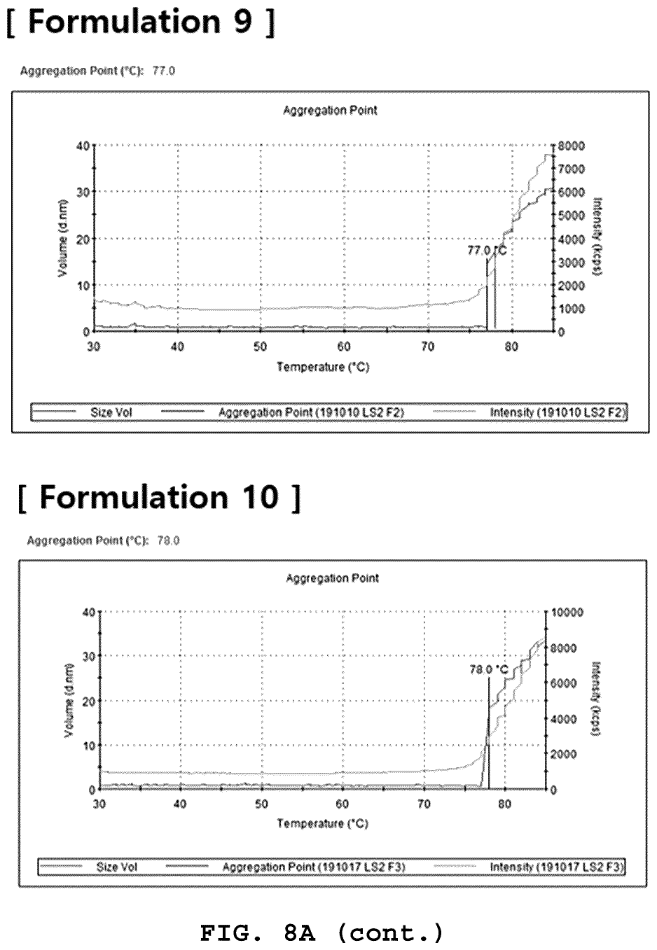

The aggregation temperature in formulation 8, not containing polysorbate 20, was 78.3° C., formulation 9 exhibited an aggregation temperature of 77.3° C., and formulation 10 exhibited an aggregation temperature of 77.7° C. In Working Example 13, no change in monomer ratio of the protein was shown despite not containing polysorbate 20, and as a result of comparing the case of not containing polysorbate 20 with the case of containing polysorbate 20, it was found that there was no difference in aggregation between proteins. These results indicate that a minimum amount of polysorbate 20 is not necessarily required for subcutaneous injection formulations of trastuzumab (see FIGS. 8A and 8B).

### Working Example 15. WCX Chromatography Analysis for Formulations Containing Trastuzumab and HP46

For WCX chromatography analysis, an HPLC system available from Shimadzu Prominence, and as columns, a TSKgel CM-STAT column (4.6×100 mm, 7 μm), a TSKgel guard gel CMSTAT (3.2 mm I.D.×1.5 cm), and the like were used. Mobile phase A is 10 mM sodium phosphate (pH 7.5) and mobile phase B is 10 mM sodium phosphate (pH 7.2) containing 0.1 M NaCl. Analysis was carried out for 55 minutes with a linear concentration gradient of 0 to 30% mobile phase B at a flow rate of 0.8 mL/min. The sample was diluted with mobile phase A so that the final concentration was 1.0 mg/mL, 80 μL of the sample was injected into HPLC, and then absorbance of a column eluate at 280 nm was recorded. The monomer ratio of trastuzumab in the HPLC chromatogram was calculated and graphed.

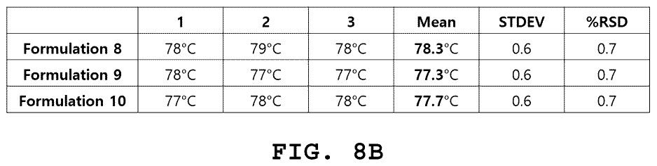

Formulations 8 to 10 showed similar change patterns when WCX analysis was performed in a stability test under harsh conditions at 40° C. for 14 days. Specific changes include an increase in the relative content of acidic variants (approximately 10% change for 14 days), a decrease in the relative content of main peaks (approximately 40% change for 14 days), and an increase in the relative content of basic variants (approximately 300% change for 14 days), and there was no significant difference according to formulation. In conclusion, in the WCX analysis in a stability test under harsh conditions at 40° C., protein stability according to polysorbate 20 (0 to 0.04%) was similar (see FIGS. 9A-9D). Working Example 16. Measurement of Enzymatic Activity for Formulations Containing Trastuzumab and HP46

The turbidimetric assay method for measuring enzymatic activity is a method of measuring, by absorbance, the degree to which an aggregate is formed by binding of residual hyaluronic acid to acidified albumin (BSA), and when hyaluronic acid is hydrolyzed by PH20, the extent of binding to albumin is reduced, resulting in reduced absorbance. BTH (Sigma) as a standardized product was diluted to 1, 2, 5, 7.5, 10, 15, 20, 30, 50, and 60 units/mL and prepared in each tube. Purified PH20 variant samples were diluted with enzyme diluent buffer (20 mM Tris·HCl, pH 7.0, 77 mM NaCl, 0.01% (w/v) bovine serum albumin) to 100×, 300×, 600×, 1200×, and 2400× and prepared in each tube. In fresh tubes, the hyaluronic acid solution, having a concentration of 3 mg/mL, was diluted 10-fold to a concentration of 0.3 mg/mL so that the volume of each tube became 180 μL. 60 μL of the sample containing hyaluronidase was added to the diluted hyaluronic acid solution and mixed therewith, and allowed to react at 37° C. for 45 minutes. After the reaction was completed, 50 μL of the reacted enzyme and 250 μL of an acidic albumin solution were added to each well of a 96-well plate and shaken for 10 minutes, and then absorbance at 600 nm was measured using a spectrophotometer.

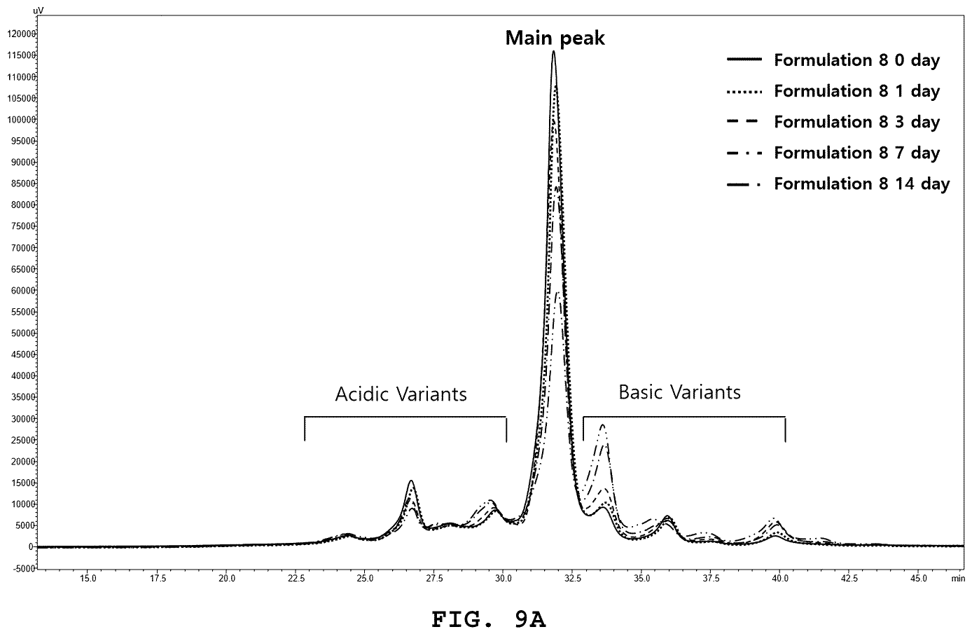

As a result of performing activity analysis in a stability test under harsh conditions at 40° C. for 14 days, it was found that the higher the concentration of polysorbate 20, the greater the reduction in activity over time (see FIG. 10).

### Working Example 17. Formulation Development

Three trastuzumab subcutaneous injection formulations were prepared as shown in Table 9. Formulations 11 to 13 commonly contain 120 mg/mL of trastuzumab, 20 mM histidine/histidine-HCl (pH 5.5), 210 mM trehalose, 10 mM methionine, and a PH20 variant. The difference among formulations 11 to 13 is the concentration of a nonionic surfactant, wherein formulation 11:0% polysorbate 80, formulation 12:0.005% polysorbate 80, and formulation 13:0.04% polysorbate 80.

TABLE 9
Composition of formulations

| | Formulation | Formulation | Formulation |
|---|---|---|---|
| | 11 | 12 | 13 |
| Antibody | Trastuzumab (120 mg/mL) | | |
| Polysorbate 80 | 0% | 0.005% | 0.04% |

| | |
|---|---|
| Buffer | 20 mM histidine/histidine-HCl |
| Stabilizer 1 | 210 mM trehalose |
| Stabilizer 2 | 10 mM methionine |
| pH | 5.5 |
| Hyaluronidase | HP46 of SEQ ID NO: 44 (2,000 units/mL) |

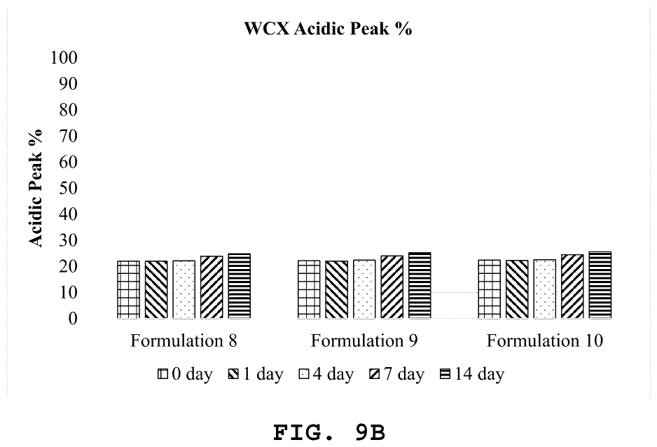

When size-exclusion chromatography analysis was performed in a stability test under harsh conditions at 40° C. for 14 days, formulations 11 to 13 showed similar change patterns. The major changes were increases in high-molecular-weight (HMW) and low-molecular-weight (LMW) degradation products and a decrease in monomer content (about less than 1.0%), and there was no significant difference according to formulation. In conclusion, as a result of performing size-exclusion chromatography analysis in a stability test under harsh conditions at 40° C., there was no significant difference in stability profile between the formulations according to the concentration (0 to 0.04%) of polysorbate 80 (see FIG. 11).

### Example 18. WCX Chromatography Analysis for Formulations Containing Trastuzumab and HP46

For WCX chromatography analysis, an HPLC system available from Shimadzu Prominence and as columns, a TSKgel CM-STAT column (4.6×100 mm, 7 μm), a TSKgel guard gel CMSTAT (3.2 mm i.d.×1.5 cm), and the like were used. Mobile phase A is 10 mM sodium phosphate (pH 7.5) and mobile phase B is 10 mM sodium phosphate (pH 7.2) containing 0.1 M NaCl. Analysis was carried out for 55 minutes with a linear concentration gradient of 0 to 30% mobile phase B at a flow rate of 0.8 mL/min. The sample was diluted with mobile phase A so that the final concentration was 1.0 mg/mL, 80 μL of the sample was injected into HPLC, and then absorbance of a column eluate at 280 nm was recorded. The monomer ratio of trastuzumab in the HPLC chromatogram was calculated and graphed.

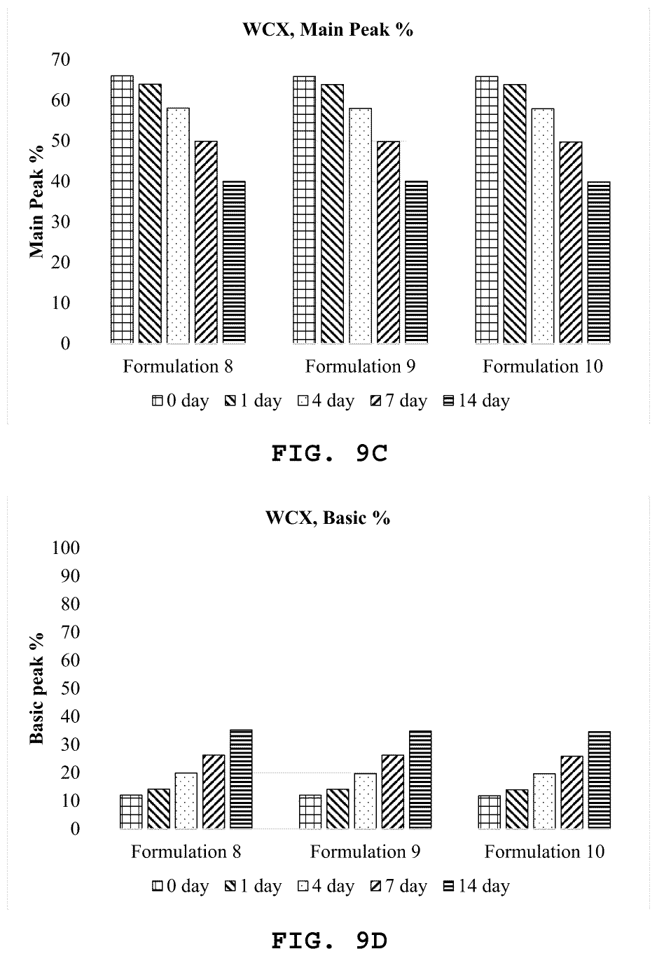

Formulations 11 to 13 showed similar change patterns when WCX analysis was performed in a stability test under harsh conditions at 40° C. for 14 days. Specific changes include an increase in the relative content of acidic variants (approximately 10% change for 14 days), a decrease in the relative content of main peaks (approximately 40% change for 14 days), and an increase in the relative content of basic variants (approximately 300% change for 14 days), and there was no significant difference according to formulation. In conclusion, in the WCX analysis in a stability test under harsh conditions at 40° C., protein stability according to polysorbate 80 (0 to 0.04%) was similar (see FIGS. 12A-12D).

### Example 19. Measurement of Enzymatic Activity for Formulations Containing Trastuzumab and HP46

Turbidimetric assay for measuring enzymatic activity is a method of measuring, by absorbance, the degree to which an aggregate is formed by binding of residual hyaluronic acid to acidified albumin (BSA), and when hyaluronic acid is hydrolyzed by PH20, the extent of binding to albumin is reduced, resulting in reduced absorbance. BTH (Sigma) as a standardized product was diluted to 1, 2, 5, 7.5, 10, 15, 20, 30, 50, and 60 units/mL and prepared in each tube. Purified protein samples were diluted with enzyme diluent buffer (20 mM Tris·HCl, pH 7.0, 77 mM NaCl, 0.01% (w/v) bovine serum albumin) to 100×, 300×, 600×, 1200×, and 2400× and prepared in each tube. In fresh tubes, the hyaluronic acid solution, having a concentration of 3 mg/mL, was diluted 10-fold to a concentration of 0.3 mg/mL so that the volume of each tube became 180 μL. 60 μL of the sample containing hyaluronidase was added to the diluted hyaluronic acid solution, mixed therewith, and allowed to react at 37° C. for 45 minutes. After the reaction was completed, 50 μL of the reacted enzyme and 250 μL of an acidic albumin solution were added to each well of a 96-well plate and shaken for 10 minutes, and then absorbance at 600 nm was measured using a spectrophotometer.

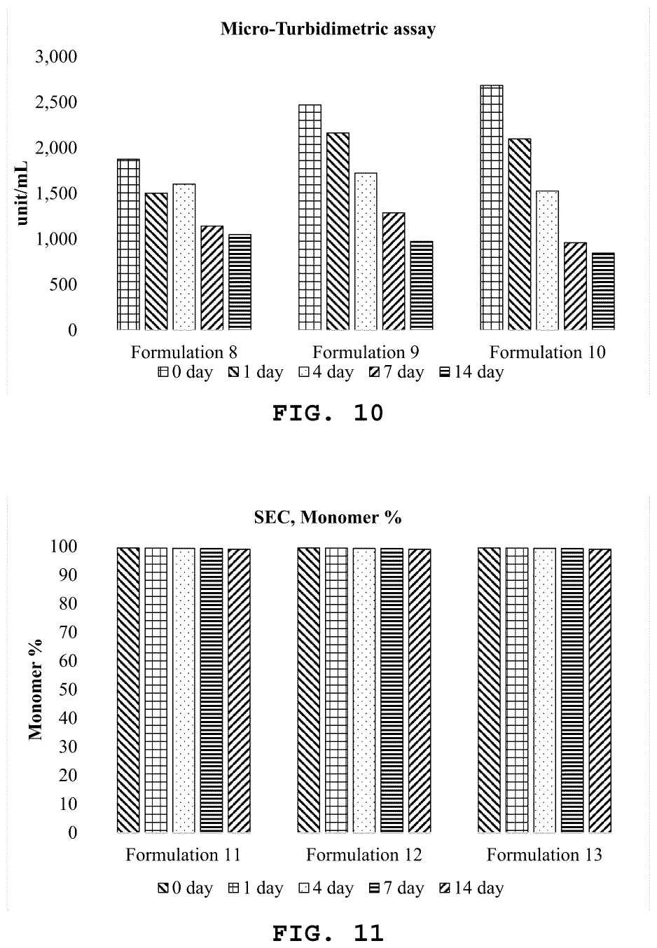

As a result of performing activity analysis in a stability test under harsh conditions at 40° C. for 14 days, it was found that the higher the concentration of polysorbate 80, the greater the reduction in activity over time (see FIG. 13).

### Working Example 20. Formulation Development

Three types of rituximab formulations were prepared as described in Table 10. Formulations 14 to 16 commonly include 120 mg/mL of rituximab, 20 mM histidine/histidine-HCl (pH 5.5), 210 mM trehalose, 10 mM methionine, and a PH20 variant. The difference among formulations 14 to 16 is the concentration of a non-ionic surfactant, which includes: formulation 1:0% polysorbate 80, formulation 2:0.005% polysorbate 80, and formulation 3:0.06% polysorbate 80.

TABLE 10
Composition of formulations

| | Formulation | Formulation | Formulation |
|---|---|---|---|
| | 14 | 15 | 16 |
| Rituximab | 120 mg/mL (±10) | | |
| PS 80 | 0% | 0.005% | 0.06% |

| | |
|---|---|
| Buffer | 20 mM histidine/histidine-HCl |
| Stabilizer 1 | 210 mM trehalose |
| Stabilizer 2 | 10 mM methionine |
| pH | 5.5 |
| Hyaluronidase | HP46 of SEQ ID NO: 44 (2,000 units/mL) |

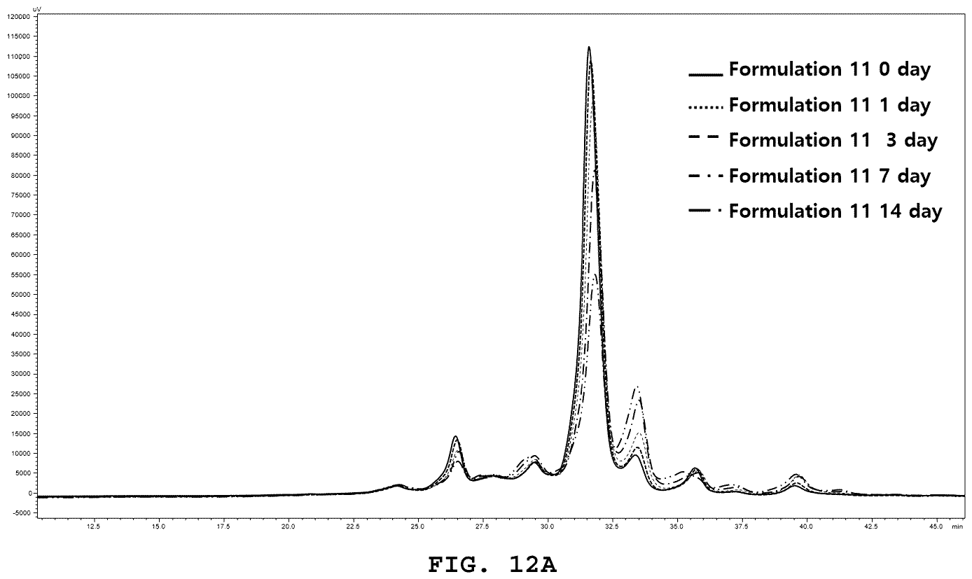

When size-exclusion chromatography analysis was performed in a stability test under harsh conditions at 40° C. for 7 days, formulations 14 to 16 showed similar change patterns. The major changes were increases in high-molecular-weight (HMW) and low-molecular-weight (LMW) degradation products and a decrease in monomer content (less than about 1.0%), and there was no significant difference according to formulation. In conclusion, as a result of performing size-exclusion chromatography analysis in a stability test under harsh conditions at 40° C., there was no significant difference in stability profile between the formulations according to the concentration (0 to 0.06%) of polysorbate 80 (see FIG. 14).

### Working Example 21. Measurement of Enzymatic Activity for Formulations Containing Rituximab and HP46

Turbidimetric assay for measuring enzymatic activity is a method of measuring, by absorbance, the degree to which an aggregate is formed by binding of residual hyaluronic acid to acidified albumin (BSA), and when hyaluronic acid is hydrolyzed by PH20, the extent of binding to albumin is reduced, resulting in reduced absorbance. BTH (Sigma) as a standardized product was diluted to 1, 2, 5, 7.5, 10, 15, 20, 30, 50, and 60 units/mL and prepared in each tube. Purified protein samples were diluted with enzyme diluent buffer (20 mM Tris·HCl, pH 7.0, 77 mM NaCl, 0.01% (w/v) bovine serum albumin) to 100×, 300×, 600×, 1200×, and 2400× and prepared in each tube. In fresh tubes, the hyaluronic acid solution, having a concentration of 3 mg/ml, was diluted 10-fold to a concentration of 0.3 mg/mL so that the volume of each tube became 180 μL. 60 μL of the sample containing hyaluronidase was added to the diluted hyaluronic acid solution, mixed therewith, and allowed to react at 37° C. for 45 minutes. After the reaction was completed, 50 μL of the reacted enzyme and 250 μL of an acidic albumin solution were added to each well of a 96-well plate and shaken for 10 minutes, and then absorbance at 600 nm was measured using a spectrophotometer.

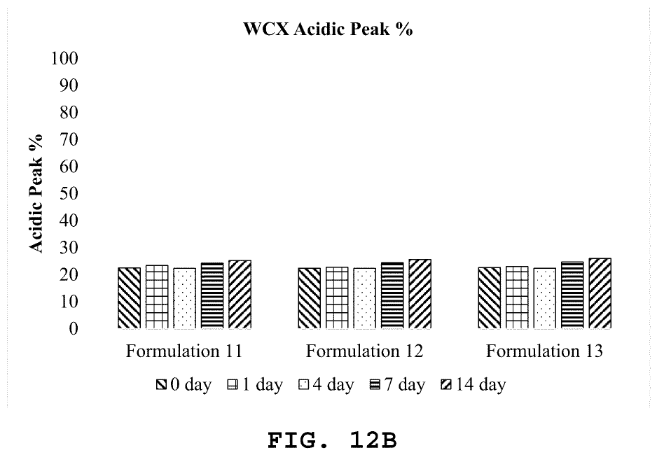

As a result of performing activity analysis in a stability test under harsh conditions at 40° C. for 7 days, it was found that the higher the concentration of polysorbate 80, the greater the reduction in activity over time (see FIG. 15).

### Working Example 22. Measurement of Enzymatic Activity in Formulations of Commercially Available Products not Containing Polysorbate

Two types of commercially available rituximab formulations were prepared as described in Table 11. Formulation 17 is a commercially available buffer for subcutaneous injection formulations, and formulation 18 is a commercially available buffer for intravenous injection formulations. Formulations 17 and 18 contain a PH20 variant and rituximab at 120 mg/mL and 100 mg/mL, respectively, but do not contain polysorbate 80 unlike formulations of commercially available products.

TABLE 11
Composition of formulations

| | Formulation | | Formulation | |
|---|---|---|---|---|
| | 17 | | 18 | |
| Rituximab | 120 | mg/mL | 100 | mg/mL |
| Buffer | 20 | mM | 25 | mM Sodium citrate |
| | histidine/histidine-HCl | | | |
| Stabilizer 1 | 210 | mM trehalose | 145 | mM NaCl |
| Stabilizer 2 | 10 | mM methionine | 10 | mM methionine |

| | | |
|---|---|---|
| pH | 5.5 | 6.5 |

| | |
|---|---|
| Hyaluronidase | HP46 of SEQ ID NO: 44 (2,000 units/mL) |

Turbidimetric assay for measuring enzymatic activity is a method of measuring, by absorbance, the degree to which an aggregate is formed by binding of residual hyaluronic acid to acidified albumin (BSA), and when hyaluronic acid is hydrolyzed by PH20, the extent of binding to albumin is reduced, resulting in reduced absorbance. BTH (Sigma) as a standardized product was diluted to 1, 2, 5, 7.5, 10, 15, 20, 30, 50, and 60 units/mL and prepared in each tube. Purified protein samples were diluted with enzyme diluent buffer (20 mM Tris·HCl, pH 7.0, 77 mM NaCl, 0.01% (w/v) bovine serum albumin) to 100×, 300×, 600×, 1200×, and 2400× and prepared in each tube. In fresh tubes, the hyaluronic acid solution, having a concentration of 3 mg/mL, was diluted 10-fold to a concentration of 0.3 mg/mL so that the volume of each tube became 180 μL. 60 μL of the sample containing hyaluronidase was added to the diluted hyaluronic acid solution, mixed therewith, and allowed to react at 37° C. for 45 minutes. After the reaction was completed, 50 μL of the reacted enzyme and 250 μL of an acidic albumin solution were added to each well of a 96-well plate and shaken for 10 minutes, and then absorbance at 600 nm was measured using a spectrophotometer.

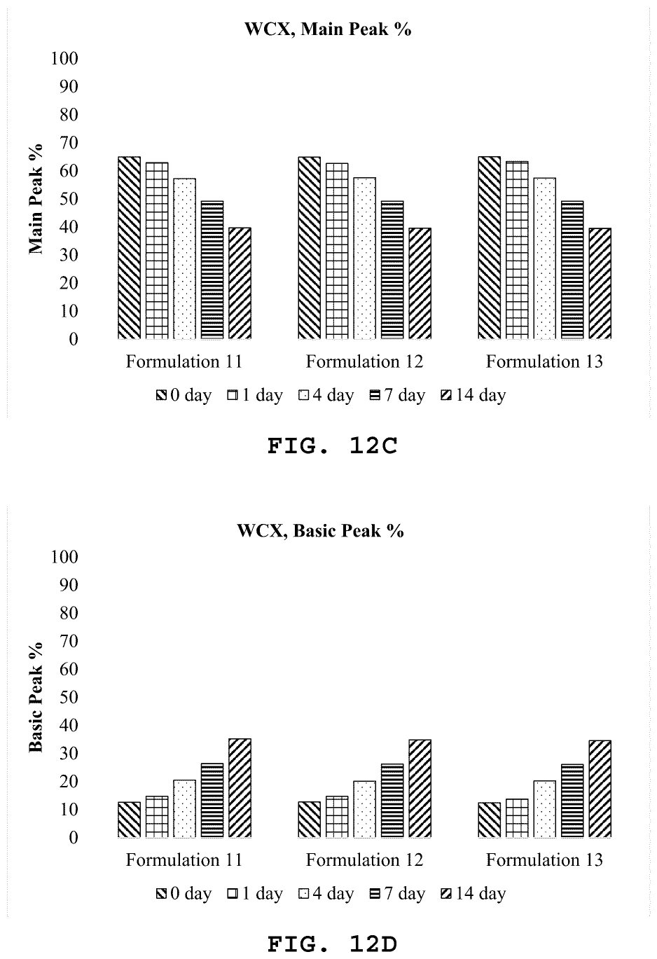

As a result of performing activity analysis in a stability test under harsh conditions at 40° C. for 6 days, it was found that high activity was maintained even in the formulations not containing polysorbate 80, and particularly, formulation 18 maintained high activity (see FIG. 16).

### Working Example 23: Formulation Development

Four types of pembrolizumab subcutaneous injection formulations were prepared as described in Table 12. Formulations 19, 20, and 21 commonly include 25 mg/mL of pembrolizumab, 10 mM histidine (pH 5.5), 7% sucrose, 10 mM methionine, and a PH20 variant. The difference among formulations 19 to 21 is the concentration of a non-ionic surfactant: formulation 19: 0% polysorbate 80, formulation 20:0. 005% polysorbate 80, and formulation 21:0.02% polysorbate 80. Formulation 22 contains 25 mg/mL of pembrolizumab and consists of 10 mM histidine (pH 5.5), 210 mM trehalose, 10 mM methionine, 0.02% polysorbate 80, and a PH20 variant.

TABLE 12
Composition of formulations

| | Formulation | Formulation | Formulation | Formulation |
|---|---|---|---|---|
| | 19 | 20 | 21 | 22 |
| Antibody | Pembrolizumab (25 mg/mL) | | | |
| Buffer | 10 mM histidine (pH 5.5) | | | |
| Stabilizer 1 | 7% | 7% | 7% | 210 mM |
| | sucrose | sucrose | sucrose | trehalose |
| Stabilizer 2 | 10 mM | 10 mM | 10 mM | 10 mM |
| | methionine | methionine | methionine | methionine |
| Polysorbate 80 | 0% | 0.005% | 0.02% | 0.02% |

| | |
|---|---|
| Hyaluronidase | HP46 of SEQ ID NO: 44 (2,000 units/mL) |

### Working Example 24. Measurement Using Spectrophotometer

Formulations 19, 20, 21, and 22 were left alone for 7 days at 40° C., and changes in protein concentration were analyzed using a spectrophotometer manufactured by Beckman. Each sample was diluted with distilled water so that the concentration of the sample was 0.4 mg/mL, and then absorbance of the protein at 280 nm was measured using a spectrophotometer.

In a stability test under harsh conditions at 40° C. for 7 days, there was no significant difference in protein concentrations of formulations 19 to 22.

### Working Example 25. Investigation of Monomer Ratio of Pembrolizumab in Each Formulation Using Size-Exclusion Chromatography

For size-exclusion chromatography analysis, an HPLC system available from Shimadzu Prominence and as columns, a TSK-gel G3000SWXL (7.8×300 mm, 5 μm) and a TSK guard column (6.0×4.0 mm, 7 μm) were used. As a mobile phase, 0.2 M potassium phosphate (pH 6.2) containing 0.25 M potassium chloride was used. Analysis was performed for 35 minutes by applying an isocratic separation mode at a flow rate of 0.5 mL/min. The sample was diluted with an analytical solvent so that the final concentration was 10 mg/mL, and after injecting 20 μL of the sample into the HPLC column, absorbance of the column eluate at 280 nm was measured. The monomer ratio of pembrolizumab in the HPLC chromatogram was calculated and graphed.

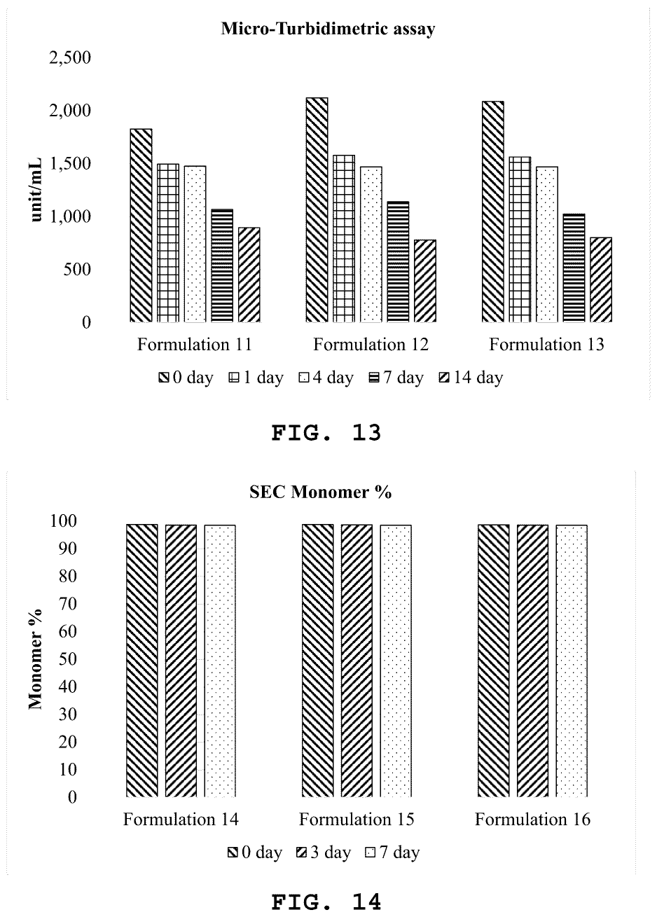

When size-exclusion chromatography analysis was performed in a stability test under harsh conditions at 40° C. for 7 days, formulations 19, 20, 21, and 22 showed similar change patterns. There was no significant difference according to formulation in the change patterns of high-molecular-weight (HMW) and low-molecular-weight (LMW) degradation products. In conclusion, as a result of performing size-exclusion chromatography analysis in a stability test under harsh conditions at 40° C., formulations 19, 20, 21, and 22 did not show any significant difference, and there was also no difference according to the type of sugar (see FIG. 17). These results were consistent with those of the cases of trastuzumab and rituximab according to the previous examples.

### Working Example 26. Measurement of Enzymatic Activity for Formulations Containing Pembrolizumab and HP46

A turbidimetric assay for measuring enzymatic activity is a method of measuring, by absorbance, the extent to which an aggregate is formed by binding of residual hyaluronic acid to acidified albumin (BSA), and when hyaluronic acid is hydrolyzed by PH20, the extent of binding to albumin is reduced, resulting in reduced absorbance. BTH (Sigma) as a standardized product was diluted to 1 unit/mL, 2, 5, 7.5, 10, 15, 20, 30, 50, and 60 units/mL and prepared in each tube. Purified protein samples were diluted with enzyme diluent buffer (20 mM Tris·HCl, pH 7.0, 77 mM NaCl, 0.01% (w/v) bovine serum albumin) to 100×, 300×, 600×, 1200×, and 2400× and prepared in each tube. In fresh tubes, the hyaluronic acid solution, having a concentration of 3 mg/mL, was diluted 10-fold to a concentration of 0.3 mg/mL so that the volume of each tube became 180 μL. 60 μL of the enzyme was added to the diluted hyaluronic acid solution, mixed therewith, and allowed to react at 37° C. for 45 minutes. After the reaction was completed, 50 μL of the reacted enzyme and 250 μL of an acidic albumin solution were added to each well of a 96-well plate and shaken for 10 minutes, and then absorbance at 600 nm was measured using a spectrophotometer.

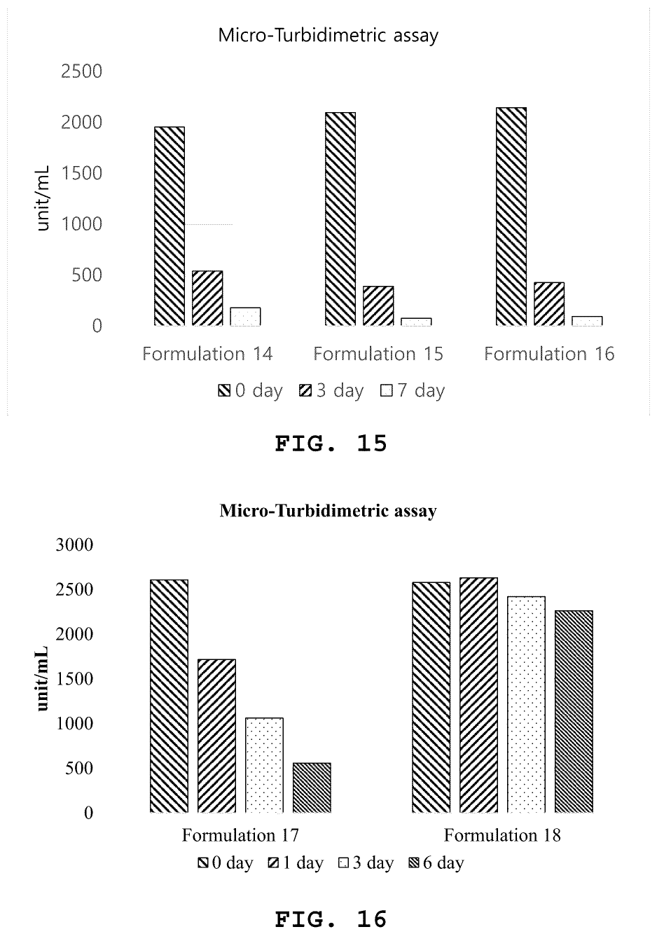

As a result of performing activity analysis in a stability test under harsh conditions at 40° C. for 7 days, it was found that the higher the concentration of polysorbate 80 is, the reduction in activity over time was somewhat large. It was also found that, when the same amount of polysorbate 80 was included, the reduction in activity was smaller in a trehalose-containing formulation than in a sucrose-containing formulation (see FIG. 18).

### Working Example 27. pH-Activity Profiles of HP46 and Wild-Type HW2

For an experiment for determining the pH-activity profiles of HP46 and wild-type HW2, a microturbidimetric assay method was used. A hyaluronic acid buffer for dissolving hyaluronic acid as a substrate and an enzyme buffer for diluting the enzyme were prepared for each pH.

A total of three 96-well plates were prepared for a reaction between the enzyme and the substrate and designated as A, B, and C, and an experiment was carried out.

A hyaluronic acid buffer at a pH level of 4.0, 4.5, or 5.0 was prepared using 20 mM acetic acid and 70 mM NaCl, and a hyaluronic acid buffer at a pH level of 5.5, 6.0, 6.5, 7.0, or 8.0 was prepared using 20 mM sodium phosphate and 70 mM NaCl. 20 mg of hyaluronic acid was dissolved in 10 mL of each of the prepared hyaluronic acid buffers to prepare a final hyaluronic acid substrate solution, which was then diluted with each hyaluronic acid buffer prepared according to pH to prepare 500 μL of the resultant solution to a concentration of 0.1 mg/mL, 0.25 mg/mL, 0.45 mg/mL, or 0.7 mg/mL, and 100 μL of each solution was dispensed into each well of the 96-well plate designated as A. The hyaluronic acid buffers, diluted and prepared according to concentration, were used as calibration curves for measuring the concentration of hyaluronic acid.

An enzyme buffer at a pH level of 4.0, 4.5, or 5.0 was prepared using 20 mM acetic acid, 0.01% (w/v) BSA, and 70 mM NaCl, and an enzyme buffer at a pH level of 5.5, 6.0, 6.5, 7.0, or 8.0 was prepared using 20 mM sodium phosphate, 0.01% (w/v) BSA, and 70 mM NaCl.

HP46 and wild-type HW2 enzymes were diluted with the enzyme buffer prepared according to pH to 10 units/mL, and 50 μL of the resultant solution was dispensed into each well of the 96-well plate designated as B.

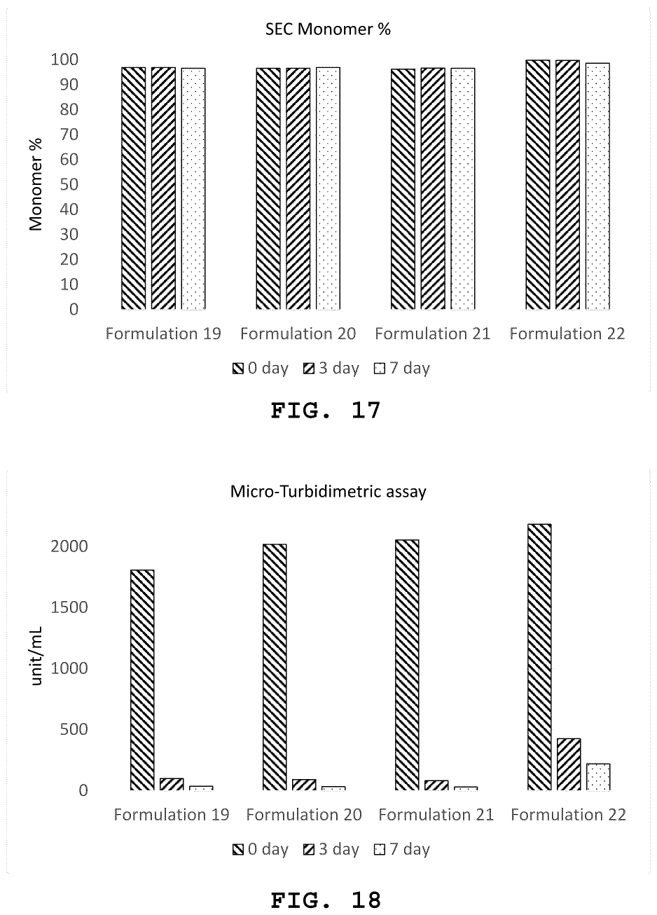

50 μL of the sample was transferred from each well of the 96-well plate designated as A to each well of the 96-well plate designated as B, followed by allowing a reaction to occur in a 37° C. shaking incubator for 45 minutes. 15 minutes before the reaction was completed, 200 μL of an acidic albumin solution was dispensed into each well of the 96-well plate designated as C and prepared, and when the enzymatic substrate reaction was completed, 40 μL of the sample was transferred from each well of the 96-well plate designated as B to each well of the 96-well plate designated as C, followed by allowing a reaction to occur for 20 minutes. After 20 minutes, absorbance at 600 nm was measured, and the amount of hyaluronic acid remaining after the enzymatic substrate reaction was calculated, and the activity profiles of the enzymes according to pH were completed (see FIG. 19).

### Working Example 28. Test for Pharmacokinetics Herceptin Subcutaneous Injection Formulation and Using Trastuzumab and HP46 in Sprague-Dawley Rats

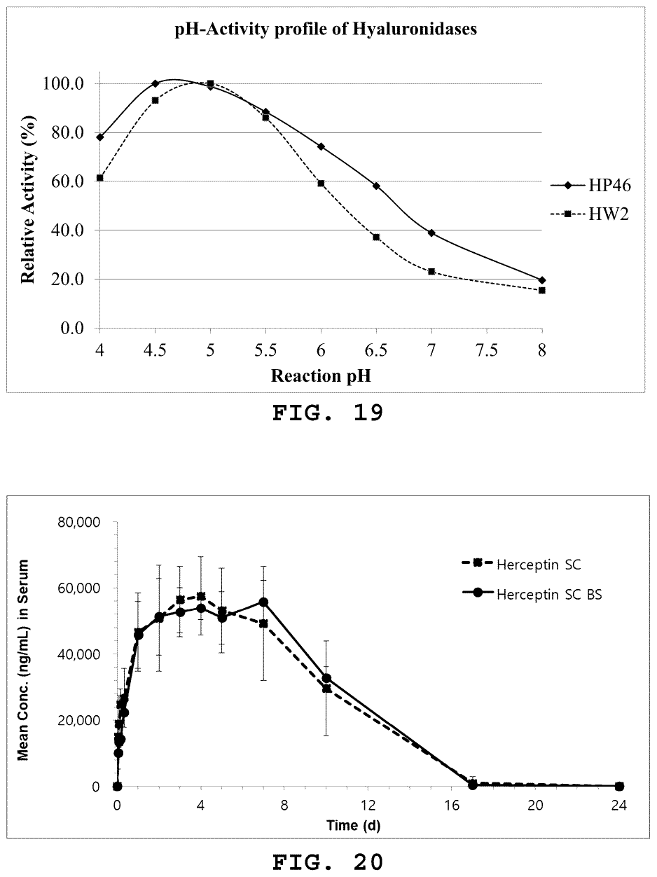

To examine whether a subcutaneous injection formulation of trastuzumab and HP46 exhibits the same pharmacokinetic properties as those of a Herceptin subcutaneous injection formulation, an experiment was conducted using 9-week-old Sprague-Dawley rats. The dose of administered Herceptin and trastuzumab was 18 mg/kg of rat body weight, the amount of rHuPH20 included in the Herceptin subcutaneous injection formulation was 100 U, and the amount of HP46 was also 100 U. In the pharmacokinetic test, trastuzumab and HP46 showed the same Area Under the Curve (AUC) as that of the Herceptin subcutaneous injection formulation (see FIG. 20).

### INDUSTRIAL APPLICABILITY

A pharmaceutical composition according to the present invention can be used for subcutaneous administration (subcutaneous injection) and is also very stable, and the activity of PH20 variants along with a drug, preferably an antibody drug or the like, can be maintained for a long time. Thus, the pharmaceutical composition can contribute to a reduction not only in the cost of producing subcutaneous injection formulations but also in medical costs, and is very advantageous in terms of convenience of patients.

### REFERENCES

Bookbinder, L. H., Hofer, A., Haller, M. F., Zepeda, M. L., Keller, G. A., Lim, J. E., Edgington, T. S., Shepard, H. M., Patton, J. S., and Frost, G. I. (2006). A recombinant human enzyme for enhanced interstitial transport of therapeutics. J Control Release 114, 230-241.
Borders jr., C. L. and Raftery, A. (1968) Purification and Partial Characterization of Testicular Hyaluronidase. J Biol Chem 243, 3756-3762.
Chao, K. L., Muthukumar, L., and Herzberg, 0. (2007). Structure of human hyaluronidase-1, a hyaluronan hydrolyzing enzyme involved in tumor growth and angiogenesis. Biochemistry 46, 6911-6920.
Chen, K. J., Sabrina, S., El-Safory, N. S., Lee, G. C., and Lee, C. K. (2016) Constitutive expression of recombinant human hyaluronidase PH20 by Pichia pastoris. J Biosci Bioeng. 122, 673-678.
Frost, G. I. (2007). Recombinant human hyaluronidase (rHuPH20): an enabling platform for subcutaneous drug and fluid administration. Expert Opin Drug Deliv 4, 427-440.
Hofinger, E. S., Bernhardt, G., and Buschauer, A. (2007) Kinetics of Hyal-1 and PH-20 hyaluronidases: comparison of minimal substrates and analysis of the transglycosylation reaction. Glycobiology 17, 963-971.
Kreidieh, F. Y., Moukadem, H. A., and Saghir, N. S. E. (2016) Overview, prevention and management of chemotherapy extravasation. World J Clin Oncol 7, 87-97.
Thomas, J. R., Yocum, R. C., Haller, M. F., and Flament J. (2009) The INFUSE-Morphine IIB Study: Use of Recombinant Human Hyaluronidase (rHuPH20) to Enhance the Absorption of Subcutaneous Morphine in Healthy Volunteers. J Pain Symptom Manag 38, 673-682.

### Sequence Listing (Free Text)

The electronic file is attached.

1. A pharmaceutical composition comprising:
(a) pembrolizumab; and
(b) a PH20 variant,
wherein the PH20 variant comprises one or more amino acid residue substitutions selected from the group consisting of S343E, M345T, K349E, L353A, L354I, N356E, and I361T, in wild-type PH20 having a sequence of SEQ ID NO: 1.

2. The pharmaceutical composition of claim 1, wherein the PH20 variant comprises one or more amino acid residue substitutions selected from the group consisting of L354I and N356E.

3. The pharmaceutical composition of claim 1, wherein the PH20 variant further comprises one or more amino acid residue substitutions in one or more regions selected from the group consisting of the regions corresponding to an alpha helix region and a linker region thereof of the wild-type PH20 of SEQ ID NO: 1.

4. The pharmaceutical composition of claim 3, wherein the alpha helix region of the wild-type PH20 of SEQ ID NO: 1 is an alpha-helix 8 region (S347 to C381), and the linker region is a linker region (A333 to R346) between alpha-helix 7 and alpha-helix 8.

5. The pharmaceutical composition of claim 4, wherein the region corresponding to the alpha-helix region and the linker region of the wild-type PH20 of SEQ ID NO: 1 is T341 to N363, T341 to I361, L342 to I361, S343 to I361, I344 to I361, M345 to I361, or M345 to N363.

6. The pharmaceutical composition of claim 4, wherein one or more regions selected from the group consisting of the alpha-helix 8 region (S347 to C381) and the linker region (A333 to R346) between alpha-helix 7 and alpha-helix 8 of the wild-type PH20 of SEQ ID NO: 1 are substituted with portion(s) of amino acid residues of the amino acid sequence of a corresponding region of Hyal1.

7. The pharmaceutical composition of claim 1, wherein the PH20 variant comprises amino acid residue substitution(s) of L354I and/or N356E, and further comprises amino acid residue substitution(s) at one or more positions selected from the group consisting of T341, L342, S343, I344, M345, S347, M348, K349, L352, L353, D355, E359, I361, and N363.

8. The pharmaceutical composition of claim 7, wherein the PH20 variant comprises amino acid residue substitution(s) of L354I and/or N356E, and further comprises one or more amino acid residue substitutions selected from the group consisting of T341S, L342W, S343E, I344N, M345T, S347T, M348K, K349E, L352Q, L353A, D355K, E359D, I361T, and N363G.

9. The pharmaceutical composition of claim 7, wherein the PH20 variant comprises amino acid residue substitutions of M345T, S347T, M348K, K349E, L352Q, L353A, L354I, D355K, N356E, E359D, and I361T.

10. The pharmaceutical composition of claim 9, wherein the PH20 variant further comprises one or more amino acid residue substitutions selected from the group consisting of T341S, L342W, S343E, I344N, and N363G.

11. The pharmaceutical composition of claim 10, wherein the PH20 variant comprises any one amino acid substitution selected from the group consisting of the following:
(a) T341S, L342W, S343E, I344N, M345T, S347T, M348K, K349E, L352Q, L353A, L354I, D355K, N356E, E359D, and I361T;
(b) L342W, S343E, I344N, M345T, S347T, M348K, K349E, L352Q, L353A, L354I, D355K, N356E, E359D, and I361T;
(c) M345T, S347T, M348K, K349E, L352Q, L353A, L354I, D355K, N356E, E359D, and I361T;
(d) M345T, S347T, M348K, K349E, L352Q, L353A, L354I, D355K, N356E, E359D, I361T, and N363G;
(e) I344N, M345T, S347T, M348K, K349E, L352Q, L353A, L354I, D355K, N356E, E359D, and I361T; and
(f) S343E, I344N, M345T, S347T, M348K, K349E, L352Q, L353A, L354I, D355K, N356E, E359D, and I361T.

12. The pharmaceutical composition according to claim 1, wherein the PH20 variant further comprises deletion of portion(s) of amino acid residues in at least one of a C-terminus and an N-terminus.

13. The pharmaceutical composition of claim 12, wherein in the PH20 variant, portion(s) of amino acid residues are deleted by truncation before an amino acid residue selected from the group consisting of M1 to P42 at the N-terminus.

14. The pharmaceutical composition of claim 13, wherein in the PH20 variant, portion(s) of amino acid residues are deleted by truncation before amino acid residue L36, N37, F38, R39, A40, P41, or P42 at the N-terminus.

15. The pharmaceutical composition of claim 12, wherein in the PH20 variant, portion(s) of amino acid residues are deleted by truncation after an amino acid residue selected from the group consisting of V455 to L509 at the C-terminus.

16. The pharmaceutical composition of claim 15, wherein in the PH20 variant, portion(s) of amino acid residues are deleted by truncation after an amino acid residue selected from the group consisting of V455 to S490 at the C-terminus.

17. The pharmaceutical composition of claim 16, wherein in the PH20 variant, portion(s) of amino acid residues are deleted by truncation after amino acid residue V455, C458, D461, C464, I465, D466, A467, F468, K470, P471, P472, M473, E474, T475, E476, P478, 1480, Y482, A484, P486, T488, or S490 at the C-terminus.

18. (canceled)

19. The pharmaceutical composition according to claim 1, wherein the PH20 variant has an amino acid sequence selected from the amino acid sequences of SEQ ID NO: 5 to SEQ ID NO: 50.

20. The pharmaceutical composition of claim 19, wherein the PH20 variant has the sequence of SEQ ID NO: 44.

21.-26. (canceled)

27. The pharmaceutical composition of claim 1, which further comprises one or more selected from the group consisting of a buffer, a stabilizer, and a surfactant.

28.-32. (canceled)

33. An injection formulation for subcutaneous administration, the injection formulation comprising the pharmaceutical composition according to claim 1.
